(19) United States
(12) Patent Application Publication
(10) Pub. No.: US 2022/0323409 A1
(43) Pub. Date: Oct. 13, 2022

(54) MODIFIED RELEASE FORMULATIONS AND USES THEREOF

(71) Applicant: SUNOVION PHARMACEUTICALS INC., Marlborough, MA (US)

(72) Inventors: Takumi ASADA, Osaka (JP); Gerald R. GALLUPPI, Marlborough, MA (US); Seth Cabot HOPKINS, Marlborough, MA (US); Megumi MARUYAMA, Osaka (JP); Siriporn TOONGSUWAN, Marlborough, MA (US); Yuki TSUSHIMA, Osaka (JP)

(21) Appl. No.: 17/616,460

(22) PCT Filed: Jun. 4, 2020
(86) PCT No.: PCT/US2020/036084

§ 371 Date: Dec. 3, 2021

Related U.S. Application Data
(60) Provisional application No. 63/011,584, filed on Apr. 17, 2020.
(60) Provisional application No. 62/856,863, filed on Jun. 4, 2019.

(51) Int. Cl. A61K 31/40; A61P 25/18; A61K 9/20

(52) U.S. Cl. CPC A61K 31/40; A61P 25/18; A61K 9/2018; A61K 9/2027; A61K 9/2054

(57) ABSTRACT

Provided are modified release compositions in a solid oral dosage form comprising racemic amisulpride, or pharmaceutically acceptable salts thereof, and medicaments comprising the same used for the treatment of various diseases and disorders, and methods of using same for the treatment of various diseases and disorders, including, but not limited to, dosage regimens.

### FIELD OF THE INVENTION

The present disclosures relate to modified release pharmaceutical compositions of racemic amisulpride and methods and uses thereof.

### BACKGROUND

Amisulpride is a member of the chemical class benzamide, and has the chemical name 4-amino-N-[(1-ethylpyrrolidin-2-yl)methyl]-5-ethylsulfonyl-2-methoxy-benzamide. The chemical structure of amisulpride is as follows:

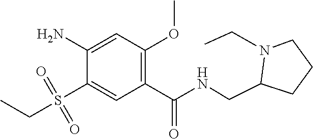 [SMILES: CCN1CCCC1CNC(=O)c1cc(S(=O)(=O)CC)c(N)cc1OC]

There is a need for better treatments of psychiatric and mood disorders, including schizophrenia, and depression.

Dopamine receptor antagonists are one class of drugs used to treat psychiatric disorders, however efficacious D₂ occupancy levels are also related to deleterious side effects. A need also therefore exists for central nervous system drugs (CNS) and in particular psychiatric drugs for the treatment of depression and diseases and disorders with a depressive component, that provide a therapeutic effect with no or reduced side effects and in particular side effects associated with dopamine D₂ receptor occupancy.

Racemic amisulpride is sold under the tradename Solian® as 400 mg tablet and as a solution for the treatment of acute and chronic schizophrenic disorders, in which positive symptoms (such as delusions, hallucinations, thought disorders) and/or negative symptoms (such as blunted affect, emotional and social withdrawal) are prominent, including patients characterized by predominant negative symptoms, with a recommended total daily dose of 400-800 mg. However, movement related adverse events including tremor, rigidity, hypokinesia, hypersalivation, akathisia, dyskinesia are listed as “very common” in the label for racemic amisulpride in the 400-800 mg/day dosage range. Such extrapyramidal symptoms are commonly associated with antipsychotic drugs employing dopamine receptor blockade. Typically, extrapyramidal symptoms are observed at high dopamine receptor occupancy, e.g., at about 70-75% occupancy.

Other side effects associated with amisulpride include prolongation of the QT interval and increase in prolactin which may lead to galactorrhoea, amenorrhoea, gynaecomastia, breast pain, erectile dysfunction. The QT interval represents the duration of ventricular depolarization and subsequent repolarization. QT interval prolongation creates an electrophysiological environment that favors the development of ventricular tachyarrhythmias, the most clinically significant being Torsades de Pointes (TdP) which can lead to ventricular fibrillation and sudden cardiac death. Patients taking one or more than one QT prolonging drug concomitantly, have an enhanced risk of TdP. Therefore, there is need for better psychiatric drugs and drug formulations with reduced side effects such as QT interval prolongation.

Thus there is a need for an amisulpride composition which has reduced adverse events and a greater safety profile.

### SUMMARY

These and other objectives make use of the unexpected discovery by the inventors of modified release formulations of racemic amisulpride compositions that provide a therapeutic effect that is the substantially the same as that of an immediate release formulation of the same amisulpride dosage, but with reduced side effects. In various aspects and embodiments, provided are modified release formulations of amisulpride with reduced drug induced QT prolongation compared to immediate release formulations having the same total amount of amisulpride.

As used herein, the terms “AUC”, “Cmax”,“Cmin”,“Tmax”, and “QT interval prolongation”, unless stated otherwise, when used in the descriptions herein encompass average, mean, and geometric mean values. That is for the sake of conciseness in description phrasing such as “average, mean, and/or geometric mean values” has not been included as it is to be understood the disclosures herein are generally applicable, mutatis mutandis.

In various aspects and embodiments, provided are modified release formulations of racemic amisulpride compositions that provide a therapeutic effect at lower total amounts of amisulpride than immediate release formulations with substantially the same D₂ dopamine receptor antagonism and 5-HT₇serotonin receptor antagonism. In various aspects and embodiments, provided are modified release formulations of racemic amisulpride compositions with reduced drug induced QT prolongation compared to immediate release formulations with substantially the same D₂ dopamine receptor antagonism and 5-HT₇ serotonin receptor antagonism.

The present inventors have discovered that the presence of amisulpride enantiomers in a subject's blood plasma is shorter than the brain D₂ dopamine receptor occupancy. The present inventors have also discovered modified release pharmaceutical formulations of amisulpride that can achieve the same brain D₂ dopamine receptor occupancy, but at lower amisulpride blood plasma concentrations (e.g. Cmax, AUC, and both Cmax and AUC), than immediate release formulations with comparable brain D₂ dopamine receptor occupancy.

In addition, the present inventors have discovered modified release pharmaceutical formulations of amisulpride that improve the therapeutic index of amisulpride. For example, in various aspects and embodiments, the present inventors have discovered modified release pharmaceutical formulations of amisulpride that provide the substantially similar pharmacodynamics (e.g. efficacy) as immediate release formulations but with improved pharmacokinetics (e.g. lower Cmax) and/or reduced side effects (e.g. reduced QT prolongation).

It has been previously discovered that the R and S amisulpride isomers have different properties. The R isomer is a selective serotonin antagonist. In contrast the S isomer is a highly selective D₂ dopamine antagonist. The present inventors provide modified release formulations using amisulpride compositions tailored to provide specific antagonism effects against the D₂ dopamine receptors and the 5-HT₇ receptors independent of one another. In various aspects and embodiments, the amisulpride compositions used in the modified release formulations have been previously shown in immediate release formulations to provide the ability to adjust the D₂ dopamine and 5-HT₇ receptors antagonism activity and reduce the adverse effects associated with racemic amisulpride of comparable total dosage amounts. The modified release formulations reduce even further the adverse effects associated with racemic amisulpride of comparable total dosage amounts. In short, the present inventors have discovered modified release formulations of these racemic amisulpride compositions to substantially the same benefits in the treatment of schizophrenia and depression as comparable immediate release formulations of these racemic amisulpride compositions but with reduced side effects in various embodiments.

In various aspects and embodiments, the racemic amisulpride compositions used in the modified release formulations provide the ability to adjust release of the active pharmaceutical ingredients (i.e. enantiomers of amisulpride) such that the D₂ dopamine and 5-HT₇ receptors antagonism activity (associated, respectively, with S amisulpride and R amisulpride) can be achieved at lower blood concentration levels than for comparable immediate release formulations of comparable total dosage amounts. Thus, in various aspects and embodiments, the modified release formulations reduce the adverse effects associated with comparable immediate release formulations of the comparable racemic amisulpride compositions, and reduce even further the adverse effects associated with racemic amisulpride of comparable total dosage amounts. Adverse effects associated with racemic amisulpride include, but are not limited to, Extrapyramidal Symptoms (EPS), akathisia, sedation, metabolic parameters such as weight gain, glucose and lipids, prolactin related events, sexual dysfunction and manic depression. Adverse effects associated with both amisulpride enantiomers include, but are not limited to, QT prolongation. In various aspects and embodiments, the degree of reduction is determined by the decrease in Cmax.

In various aspects and embodiments, provided are various modified release formulations, methods and medicaments comprising and/or employing racemic amisulpride, or pharmaceutically acceptable salts thereof, that can decreasing the undesirable side effects associated with immediate release formulations of amisulpride. In various aspects and embodiments, modified release formulations decrease the undesirable side effects associated with higher levels of dopamine D2 receptor blockade associated with (S)-(-)-amisulpride. In various aspects and embodiments, modified release formulations decrease the undesirable amisulpride side effect of drug induced QT prolongation.

It has been discovered by the inventors thatmodified release formulations of a fixed-dose combination of amisulpride enantiomers, defined in various embodiments by the contribution of 5-HT₇ occupancy relative to D2 occupancy, exhibit clinical benefit by allowing physicians to treat subjects with a dominant 5-HT₇ pharmacodynamics while still maintaining a dose-responsive underlying dopamine D₂ activity for a combined, and in various embodiments improved, clinical benefit in depressive disorders, whilst reducing one or more side effects associated with comparable immediate release formulations.

In various aspects and embodiments, there are provided a modified release pharmaceutical compositions in a solid oral dosage form comprising racemic amisulpride, or pharmaceutically acceptable salts thereof, and one or more pharmaceutically acceptable excipients. The one or more pharmaceutically acceptable excipients may include an extended release agent.

In various aspects and embodiments, when the modified release pharmaceutical composition is administered to a subject population, it results in a maximum QT interval prolongation of less than about 0.45 milliseconds (ms), less than about 0.30 milliseconds (ms), less than about 0.20 milliseconds (ms), less than about 0.10 milliseconds (ms), less than 0.05 milliseconds (ms), or less than 0.02 milliseconds (ms) per 10 mg of amisulpride for the time period of 12 hours after administration.

In various aspects and embodiments, when the modified release pharmaceutical composition is administered to a subject population, it results in a maximum QT interval prolongation for the time period of 12 hours after administration that is at least about 75%, about 65%, about 60%, about 55%, or about 50% less than that of an immediate release composition having the same total daily amount of amisulpride as the modified release pharmaceutical composition.

In various aspects and embodiments, when the modified release pharmaceutical composition is administered to a subject population, it results in a maximum QT interval prolongation for the time period of 12 hours after administration that is at least about 75%, about 65%, about 60%, about 55%, or about 50% less than that of an immediate release formulationan immediate release formulation having the same total daily amount of amisulpride as the modified release pharmaceutical composition.

In various aspects and embodiments, the solid oral dosage form, when dissolution tested using in vitro gastrointestinal simulation dissolution test releases (a) less than about 30% of amisulpride after about 1 hour, releases more than about 20% and less than about 60% of amisulpride after about 3 hours, and releases more than about 30% and less than about 100% of amisulpride mixture after about 6 hours; (b) less than about 30% of amisulpride after about 1 hour, releases more than about 20% and less than about 60% of amisulpride after about 3 hours, and releases more than about 30% and less than about 75% of amisulpride after about 6 hours; (c) less than about 20% of amisulpride after about 1 hour, releases more than about 20% and less than about 50% of amisulpride after about 3 hours, and releases more than about 30% and less than about 75% of amisulpride after about 6 hours; (d) more than about 30% and less than about 50% of amisulpride after about 6 hours; (e) no more than about 30% of amisulpride after about 1 hour, releases between about 30% and about 75% of amisulpride after about 3 hours, and releases more than about 75% of amisulpride after about 12 hours; (f) releases less than about 30% of the amisulpride after 1 hour, releases more than 25% and less than about 55% of the amisulpride after 3 hours, and releases more than about 45% and less than about 75% of the amisulpride after 6 hours; or (g) more than about 75% of amisulpride after about 6 hours.

In various aspects and embodiments, the solid oral dosage form, when dissolution tested using the two-stage in vitro dissolution test described in Table 4 in the paddle apparatus described in United States Pharmacopeia Convention (USP) Apparatus 2 of Chapter 711 Dissolution; USP41-NF36 General Chapter <711> Dissolution releases (a) less than about 30% of amisulpride after about 1 hour, releases more than about 20% and less than about 60% of amisulpride after about 3 hours, and releases more than about 30% and less than about 100% of amisulpride mixture after about 6 hours; (b) less than about 30% of amisulpride after about 1 hour, releases more than about 20% and less than about 60% of amisulpride after about 3 hours, and releases more than about 30% and less than about 75% of amisulpride after about 6 hours; (c) less than about 20% of amisulpride after about 1 hour, releases more than about 20% and less than about 50% of amisulpride after about 3 hours, and releases more than about 30% and less than about 75% of amisulpride after about 6 hours; (d) more than about 30% and less than about 50% of amisulpride after about 6 hours; (e) no more than about 30% of amisulpride after about 1 hour, releases between about 30% and about 75% of amisulpride after about 3 hours, and releases more than about 75% of amisulpride after about 12 hours; (f) less than about 30% of amisulpride after about 1 hour, releases more than about 25% and less than about 55% of amisulpride after about 3 hours, and releases more than about 45% and less than about 75% of amisulpride after about 6 hours; or (g) more than about 75% of amisulpride after about 6 hours.

As used herein, the term “two-stage in vitro gastrointestinal simulation dissolution test” refers to an in vitro test designed to simulate the solution pH conditions of the stomach (stage 1) and small intestine (stage 2) of a human in a fasted state. The pH of the first stage is between about 1.2 to 3.5, and the pH of the second stage is between about 6 to about 7.4. The sample to be tested (e.g. tablet, capsule) is placed in the liquid medium of the first stage for about an hour (to simulate residence time in the stomach) prior to the medium being adjust to those of the second stage (to simulate transition to the higher pH environment of the small intestine). The dissolution medium is stirred during the test with a paddle apparatus, substantially in accord with either that described by the United States Pharmacopeia Convention (USP) Apparatus 2 of Chapter 711 Dissolution; USP41-NF36 General Chapter <711> Dissolution or that described by the paddle method of Japanese Pharmacopeia (JP) General test <6.10>, as harmonized with Ph.Eur. <2.9.3> and USP <711>. The paddle apparatus is operated between about 50 to about 75 rpm in both stages; and the temperature of the dissolution medium in both stages is maintained at about 37° C.

In various aspects and embodiments, when the modified release pharmaceutical composition is administered to a subject population, it is effective in minimizing fluctuations between Cmin and Cmax of amisulpride. In various aspects and embodiments, the modified release pharmaceutical compositions are effective in minimizing the difference between Cmin and Cmax of amisulpride compared to an immediate release formulationan immediate release formulation having the same total daily amount of amisulpride as the modified release pharmaceutical composition wherein the value of Cmin is that at about 9 hours after administration.

In various aspects and embodiments, the modifiedrelease pharmaceutical composition, when administered to a subject population, is effective in providing a population mean ratio of Cmax/Cmin of amisulpride that is less than about 2, less than about 1.9, or less than about 1.8, wherein the value of Cmin is that at about 9 hours after administration.

In various aspects and embodiments, when the modified release pharmaceutical composition is administered to a subject population (i) the area under the curve (AUC) of blood plasma concentration versus time of amisulpride from administration to Tmax (AUC_{0-Tmax}) is less than about 17%, less than about 16%, less than about 14%, or less than about 12% of the area under the curve from administration to 48 hours (AUC₀₋₄₈); and (ii) Tmax of amisulpride is between about 4 and about 6 hours after administration.

In various aspects and embodiments, when the modified release pharmaceutical composition is administered to a subject population, it provides a blood plasma Cmax of amisulpride that is less than about 75%, less than about 65%, less than about 60%, less than about 55%, or less than about 50% of the Cmax achieved by the immediate release having the same total daily amount of amisulpride as the modified release pharmaceutical composition. In various embodiments, an immediate release composition has the same total daily amount of (R)-(+)-amisulpride and (S)-(−)-amisulpride as in the modified release pharmaceutical composition.

In various aspects and embodiments, when the modified release pharmaceutical composition is administered to a subject population, it provides a blood plasma Cmax of amisulpride that is less than about 75%, less than about 65%, less than about 60%, less than about 55%, or less than about 50% of the Cmax achieved by an immediate release formulation having the same total daily amount of amisulpride as the modified release pharmaceutical composition. In various embodiments, the immediate release composition has the same total daily amount of (R)-(+)-amisulpride and (S)-(−)-amisulpride as in the modified release pharmaceutical composition.

In various aspects and embodiments, when the modified release pharmaceutical composition is administered to a subject population, it provides a blood plasma Cmax of amisulpride that is less than about 75%, less than about 65%, less than about 55%, or less than about 50% of the Cmax achieved by an immediate release composition having the same total daily amount of amisulpride as the modified release pharmaceutical composition, and when administered to a subject population provides a AUC from 0 to 48 hours after administration (AUC₀₋₄₈) of amisulpride that is at least about 60%, at least about 70%, or at least about 75% of the AUC₀₋₄₈ achieved by an immediate release composition having the same total daily amount of amisulpride as the modified release pharmaceutical composition.

In various aspects and embodiments, when the modified release pharmaceutical composition is administered to a subject population, it provides a blood plasma Cmax of amisulpride that is less than about 75%, less than about 65%, less than about 55%, or less than about 50% of the Cmax achieved by an immediate release formulation having the same total daily amount of amisulpride as the modified release pharmaceutical composition, and when administered to a subject population provides a AUC from 0 to 48 hours after administration (AUC₀₋₄₈) of amisulpride that is at least about 60%, at least about 70%, or at least about 75% of the AUC₀₋₄₈ achieved by an immediate release formulation having the same total daily amount of amisulpride as the modified release pharmaceutical composition.

In various aspects and embodiments, when the modified release pharmaceutical composition is administered to a subject population, it provides a AUC from 0 to 48 hours after administration (AUG-₀₋₄₈) of amisulpride that is: (a) at least about 60% of the AUC₀₋₄₈ achieved by an immediate release composition having the same total daily amount of amisulpride as the modified release pharmaceutical composition, (b) at least about 70% of the AUC₀₋₄₈ achieved by an immediate release composition having the same total daily amount of amisulpride as the modified release pharmaceutical composition, (c) at least about 75% of the AUC₀₋₄₈ achieved by an immediate release composition having the same total daily amount of amisulpride as the modified release pharmaceutical composition, and/or (d) at least about 80% of the AUC₀₋₄₈ achieved by an immediate release composition having the same total daily amount of amisulpride.

In various aspects and embodiments, when the modified release pharmaceutical composition is administered to a subject population, it provides a AUC from 0 to 48 hours after administration (AUC₀₋₄₈) of amisulpride that is: (a) at least about 60% of the AUC₀₋₄₈ achieved by an immediate release formulation having the same total daily amount of amisulpride as the modified release pharmaceutical composition, (b) at least about 70% of the AUC₀₋₄₈ achieved by an immediate release formulation having the same total daily amount of amisulpride as the modified release pharmaceutical composition, (c) at least about 75% of the AUC₀₋₄₈ achieved by an immediate release formulation having the same total daily amount of amisulpride as the modified release pharmaceutical composition, and/or (d) at least about 80% of the AUG-₀₋₄₈ achieved by an immediate release formulation having the same total daily amount of amisulpride.

These and other objects, features, and advantages of the inventions will become apparent from the following detailed description of the various aspects and embodiments of the inventions taken in conjunction with the accompanying tables and drawings.

All published documents cited herein are hereby incorporated herein by reference in their entirety.

### BRIEF DESCRIPTION OF THE DRAWINGS

FIGS. 1A-1B present various analytical in vitro data for the inhibition of radioligand binding activity by racemic amisulpride, (R)-amisulpride, and (S)-amisulpride; where FIG. 1A presents data on the % inhibition of dopamine D₂ receptor binding; FIG. 1B presents data on the % inhibition of serotonin 5-HT7 receptor binding.

FIG. 2 presents in vitro dissolution profiles for various modified release pharmaceutical matrix tablet formulations of racemic amisulpride of Table 3B.

### DETAILED DESCRIPTION

Reference in the specification to “one embodiment,” “an embodiment,” “one aspect,” or “an aspect” means that a particular, feature, structure or characteristic described in connection with the embodiment or aspect is included in at least one embodiment or aspect of the teachings.

As used herein, the recitation of “amisulpride,” unless expressly further limited, refers to amisulpride in any enantiomeric ratio including, equal mixtures of R-amisulpride and S-amisulpride, pure R-amisulpride, and pure S-amisulpride. In addition, as used herein, the recitation of “amisulpride,” unless expressly further limited, includes pharmaceutically acceptable salts of amisulpride. As used herein, the term “racemic amisulpride” refers to a substantially 50:50 mixture by weight of (R)-amisulpride and (S)-amisulpride. Racemic amisulpride can be purchased commercially or prepared by combining equal amounts of (R)-amisulpride and (S)-amisulpride.

As used herein, the phrase “QT interval” refers to the heart rate corrected QT interval as determined using Fridericia's formula QTcF=QT/∛RR, that is herein “QT interval” refers to QTcF. As used herein the phrase “QT interval prolongation” refers to the change in the QTcF interval relative to the baseline QTcF interval. i.e., (ΔQTcF).

As used herein, the term “fed state” refers to the metabolic state shortly after ingestion of a meal. Measurements of a fed state pharmacokinetic parameters, such as for example, Cmax, Tmax, AUC can be conducted as follows. Following an overnight fast of at least 10 hours, subjects consume a meal comprising 150, 250, and 400-600 calories from protein, carbohydrate, and fat, respectively. This meal should be consumed about 30 minutes prior to administration of the drug product and subjects should eat this meal in 30 minutes or less. No food should be allowed for at least 4 hours post-dose. Water can be allowed as desired except for one hour before and after drug product administration.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail J. Pharmaceutical Sciences, 1977, 66, 1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like. Although pharmaceutically acceptable counter ions will be preferred for preparing pharmaceutical formulations, other anions are quite acceptable as synthetic intermediates.

As used herein, the term “subject,” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); and mammals used for the testing of pharmaceuticals.

Unless otherwise specified, the word “includes” (or any variation thereon, e.g., “include”, “including”, etc.) is intended to be open-ended. For example, “A includes 1, 2 and 3” means that A includes but is not limited to 1, 2 and 3.

As used herein, the terms “treatment,” “treat,” and “treating” refer to alleviating, inhibiting, and/or reducing one or more signs or symptoms of a disease, condition, or disorder. In various embodiments, treatment may be administered after one or more symptoms have developed. Treatment may also be continued after symptoms have resolved, for example to prevent or delay their recurrence.

As used herein, the phrase “on a free base basis” indicates that the amount of amisulpride (R and S-amisulpride) is measured based on the molecular weight of amisulpride free base. Unless specified otherwise, the weight amount described herein for amisulpride refers to the free base.

The compounds disclosed herein can include isotopes. Isotopes include those atoms having the same atomic number but different mass numbers. For example, isotopes of hydrogen include tritium and deuterium. In some embodiments, one or more atoms of the compounds can be replaced or substituted with isotopes of the atoms in natural or non-natural abundance. In some embodiments, one or more hydrogen atoms in a compound of the present disclosure can be replaced or substituted by deuterium.

As used herein, and unless otherwise specified, the term “about”, when used in connection with a numeric value or range of values may vary by 5%, 4%, 3%, 2%, 1%, 0.9%, 0.8%, 0.7%, 0.6%, 0.5%, 0.4%, 0.3%, 0.2% or 0.1% of the recited value or range of values. In some embodiments, the numeric value or range of values vary by 5%.

As used herein, and unless otherwise specified, the term “therapeutically effective” when used in connection with the pharmaceutical compositions of the present inventions means a biological or medical response which is sought or desired, for example, by a researcher or physician, such as improved treatment, healing, prevention or elimination of a disease, syndrome, condition, complaint, disorder or side-effects or also the reduction in the advance of a disease, complaint or disorder. The term “therapeutically effective amount” when used in connection with the pharmaceutical compositions of the present inventions means an amount of a medicament or of an active pharmaceutical ingredient that is therapeutically effective. The term “therapeutically effective blood plasma concentration” when used in connection with the pharmaceutical compositions of the present inventions means an active pharmaceutical ingredient blood plasma concentration that is therapeutically effective.

Other abbreviations not explicitly described herein have their normal meanings in the art.

The present disclosures relate to modified release formulations of pharmaceutical compositions comprising racemic amisulpride, medicaments for the treatment of a disorder comprising modified release formulations of racemic amisulpride, methods of treating a disorder in a subject with modified release formulations of pharmaceutical compositions comprising racemic amisulpride, and methods of inhibiting dopamine D₂ activity and serotonin 5-HT7 activity in a subject with modified release formulations comprising racemic enantiomers.

In various aspects, the disorder which the medicaments and methods treat comprise one or more of a: psychiatric disorder;; such as schizophrenia; acute schizophrenia; chronic schizophrenia; positive symptoms of schizophrenia (e.g. delusions, hallucinations, thought disorders); negative symptoms of schizophrenia; persistent depressive disorder (PDD); anxiety disorder; obsessive-compulsive disorder; behavior disturbances associated with a neurocognitive disorder; conduct disorder; neurological disorder; medication-induced movement disorder; and motor disorder. In various aspects, the condition the medicaments and methods treat comprises postoperative nausea and vomiting.

Amisulpride has a single asymmetric center and as a result exists in two enantiomeric forms: R-4-Amino-N-[(1-ethyl-2-pyrrolidinyl)methyl]-5-(ethylsulfonyl)-2-methoxybenzamide (also referred to as: (R)-(+)-4-amino-N-[(1-ethylpyrrolidin-2-yl)methyl]-5-(ethylsulfonyl)-2-methoxybenzamide, and under the IUPAC name as 4-amino-5-(ethanesulfonyl)-N-{[(2R)-1-ethylpyrrolidin-2-yl]methyl}-2-methoxybenzamide), abbreviated herein as (R)-(+)-amisulpride or (R)-amisulpride; and S-4-Amino-N-[1-ethyl-2-pyrrolidinyl)methyl]-5-(ethylsulfonyl)-2-methoxybenzamide (also referred to as: (S)-(−)-4-amino-N-[(1-ethylpyrrolidin-2-yl)methyl]-5-(ethylsulfonyl)-2-methoxybenzamide, and under the IUPAC name as 4-amino-5-(ethanesulfonyl)-N-{[(2S)-1-ethylpyrrolidin-2-yl]methyl}-2-methoxybenzamide), abbreviated herein as (S)-(−)-amisulpride or (S)-amisulpride. These two enantiomeric forms have the following chemical structures:

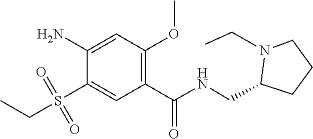 [SMILES: CCN1CCC[C@@H]1CNC(=O)c1cc(S(=O)(=O)CC)c(N)cc1OC]

### R-4-Amino-N-[(1-ethyl-2-pyrrolidinyl)methyl]-5-(ethylsulfonyl)2-methoxybenzamide, (R)-amisulpride

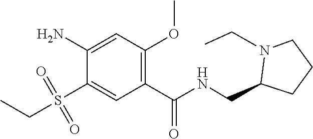 [SMILES: CCN1CCC[C@H]1CNC(=O)c1cc(S(=O)(=O)CC)c(N)cc1OC]

### S-4-Amino-N-[(1-ethyl-2-pyrrolidinyl)methyl]-5-(ethylsulfonyl)-2-methoxybenzamide (S)-amisulpride

Dopamine D₂-related side effects are well-known from clinical experience. It has been observed that the incidence of extrapyramidal side effects increases when occupancy exceeds the 80% threshold and studies have shown that extrapyramidal side effects occur even at about 70-75% occupancy (G. Grunder, et al., Nature, 8, 198-202, (2009); Nyberg, et al., Am. J. Psychiatry, 156, 873-875 (1999); Farde, et al. Arch. Gen. Psychiatry, 49, 538-544 (1992)). However, it is believed that very high D2/3 receptor occupancy is not only associated with but generally required for effectiveness against the positive symptoms of schizophrenia and that the antipsychotic effects of dopamine receptor antagonists occur within a therapeutic window between 60 and 80% striatal D2/3 receptor occupancy. (G. Grunder, et al., Nature, 8, 198-202, (2009)).

Dopamine D2-related side effects are also known from clinical experience with racemic amisulpride and include Extrapyramidal Symptoms (EPS), Tardive Dyskinesia (TD), and Akathisia. (C. Coulouvrat et al., International Clinical Psychopharmacology, Vol 14, No. 4, 209-218 (1999)). It has been determined that in general D₂ occupancy greater than about 67% results in side-effects that limit the ability of the underlying 5-HT₇ pharmacodynamics to contribute to clinical benefit as a function of dose. (Farde, et al. Arch. Gen. Psychiatry, 49, 538-544 (1992). The impact of D₂ occupancy is associated with age with EPS events being noted in older patients with Alzheimer's at occupancies of about 60%; clinically meaningful responses were seen at occupancies of 43%. (Reeves et al., Brain,140, 1117-1127). Similar results were also obtained with older patients in general. (Uchida et al., The American J. of Geriatic Pyschiatry, 22 (1) 1007-1016).

The 5-HT7 receptor has been shown, through various pharmacological tools (receptor-specific agonists and antagonists) and through the use of knockout models, to be involved in the central regulation of sleep and circadian rhythms, mood, and cognition. These same three domains are often critically impaired in mood disorders such as depressive disorder, as well as in psychotic disorders.

Provided herein are various modified release formulations of racemic amisulpride, or pharmaceutically acceptable salts thereof, can provide substantially similar or improved efficacy compared to comparable immediate release formulations whilst reducing undesired side effects, such as, for example, drug induced QT prolongation and/or those associated with higher levels of dopamine D₂ receptor blockade.

The beating of the heart is due to precisely controlled regularly spaced waves of myocardial excitation and contraction, arising from ion-based depolarization and repolarization. The electrical currents during depolarization and repolarization can be measured by leads placed on the body in specific locations (the electrocardiogram) to measure the electrical waves. The P-wave in a electrocardiogram represents a wave of depolarization in the atrium. When the entire atria becomes depolarized, the wave returns to zero, and after 0.1 seconds the ventricle is entirely depolarized resulting in the QRS complex seen in the electrocardiogram (ECG). The three peaks of the QRS complex are due to the way the current spreads in the ventricles. The QRS complex is followed by the T-wave, or repolarization of the ventricle. The QT interval is measured from the beginning of the QRS complex to the end of the T wave on the standard ECG. The QT interval represents the duration till the completion of the repolarization, phase of the cardiac myocyte (or the depolarization and repolarization of the ventricle). Prolongation of the QT interval, can result in ventricular arrhythmias, and sudden death.

Racemic amisulpride is a drug well known to induce QT interval prolongation, evidencing a substantially linear increase of prolongation with plasma concentration. (See, Taubel et al., Br. J. Clin. Pharmacology, 83, pp. 339-348 (2017)). The dangers associated with drug induced QT prolongation are also well known: “Although a QT interval of at least 500 milliseconds generally has been shown to correlate with a higher risk of Torsades de Pointes, there is no established threshold below which prolongation of the QT interval is considered free of proarrhythmic risk” (see Al-Khatib et al., JAMA, 289 (16), pp 2120-2127 (2003)). Therefore, there is need for better amisulpride formulations with reduced side effects such as QT interval prolongation.

In various aspects and embodiments, provided are various modified release formulations, methods and medicaments comprising racemic amisulpride, or pharmaceutically acceptable salts thereof, In various aspects and embodiments, the modified release formulations decrease the undesirable side effects associated with higher levels of dopamine D₂ receptor blockade associated with (S)-(−)-amisulpride. In various aspects and embodiments, the modified release formulations decrease the undesirable side effect of drug induced QT prolongation associated with racemic amisulpride.

In various aspects and embodiments, the modified release compositions are provided in a solid oral dosage form comprising racemic amisulpride, or pharmaceutically acceptable salts thereof, and one or more pharmaceutically acceptable excipients.

In various aspects and embodiments, when the modified release composition is administered to a subject population, it provides for the time period of 12 hours after administration a maximum QT interval prolongation of: (a) less than about 0.45 milliseconds (ms) per 10 mg of amisulpride; (b) less than about 0.30 milliseconds (ms) per 10 mg of amisulpride; (c) less than about 0.20 milliseconds (ms) per 10 mg of amisulpride; (d) less than about 0.15 milliseconds (ms) per 10 mg of amisulpride; (e) less than about 0.10 milliseconds (ms) per 10 mg of amisulpride; (f) less than about 0.05 milliseconds (ms) per 10 mg of amisulpride; or (g) less than about 0.02 milliseconds (ms) per 10 mg of amisulpride.

In various aspects and embodiments, the modified release compositions can reduce the population average maximum QT interval prolongation for the time period of about 12 hours after administration to a subject population relative to that for a comparable immediate release formulation.

For example, in various embodiments, the modified release compositions when administered to a subject population result in a population average maximum QT interval prolongation for the time period of about 12 hours after administration that is: (a) at least about 75%, at least about 70%, at least about 65%, at least about 60%, at least about 55%, or at least about 50% less than that of an immediate release formulation having the same total daily amount of amisulpride as the modified release composition.

A variety of methods are known to the medical art to measure a person's QT interval. The QT interval represents the duration of ventricular depolarization and subsequent repolarization. Herein, the followng method is used to determine “QT interval prolongation.” Electrocardiograms (ECGs) are recorded using a digital 12-lead Holter ECG device (for example, such as a Mortara H12+, Mortara Instruments, Milwaukee, WI) at a sampling rate of 1000 samples/second (1000 HZ). The Holter ECG recordings are started at least about 1 hour before dosing with the active pharmaceutical ingredient (API) being evaluated and continued for at least 12 hours and preferably until 24 hours after dosing. Ten ECG replicate measurements are made at least at the following time points and within 7 minutes of the time point: 45, 30, and 15 minutes before dosing (baseline) and at 1, 2, 3, 4, 6. 8, 10, and 12 hours (and optionally 24 hours) after dosing. As heart rate can affect the measurements, subjects are in a supine position during measurement.

The determination of QT interval prolongation herein for an API should exclude ECGs that exhibit morphological abnormalities, such as of the P wave, QRS complex, ST segment, T wave, U wave, rhythm and axis.

The ECGs are to be read and interpreted by a qualified cardiologist. The QT interval is measured from the initiation of the QRS complex (first deflection of the QRS complex) to the point of where the T wave returns to the isoelectric baseline. The end of the T wave is identified as the intersection of the descending part of the T wave (positive T wave) with the isoelectric line. If a U wave interrupts the T wave before it returns to baseline, the QT interval is measured as the nadir between T and U waves. If it is not clear whether the second deflection towards the descending part of the T wave is a part of the T wave or a U wave, then it is included in the QT interval. (see, e.g., Panicker GK, et al. “Intra- and interreader variability in QT interval measurement by tangent and threshold methods in a central electrocardiogram laboratory.”J Electrocardiol. 2009; 42:348-52).

The first five beats in a single lead with at least three consecutive complexes during normal rhythm, is used to measure the QT and preceding RR intervals. The PR interval and QRS duration measurements are made in the appropriate leads. Heart rate (HR) is calculated from the mean RR value. The QT interval has an inverse relationship with heart rate and shortens with increasing heart rate. As QT interval varies with change in heart rate, a heart rate correction formulae is used to transform the measure QT interval into a heart rate independent corrected value known as the QTc interval. The QTc value is intended to represent the QT interval at a standardized heart rate of 60 bpm.

The QT interval values are corrected for the effect of heart rate using the Fridericia's formula QTcF=QT/∛RR. QTcF of a given time point is calculated from the mean QT value and the mean RR interval value at that time point. QT interval prolongation is determined as the mean change from baseline values using the calculated QTcF values. Accordingly the “QT interval prolongation” at a time point is the mean QTcF change from baseline values (AQTcF).

It is to be understood that Bazett's (QTcB) formula, QTcB=QT/√RR, is another commonly used correction formula but QTcF has been chosen here for evaluation of QT prolongations instead of Bazett's formula because Bazett's formula does not adequately correct for the effect of heart rate and is known to overcorrect at high heart rates. (see, e.g., Davey P., “How to correct the QT interval for the effects of heart rate in clinical studies.” J. Pharmacol Twacol Methods. 2002; 48; 3-9). It is also to be understood that in double-blind clinical trials placebo adjusted change from baseline values of QTcF (ΔΔQTcF) however mean QTcF change from baseline values (ΔQTcF) have been chosen for use here as they do not require a double-blind protocol to determine and ECG measurements during normal clinical visits do not make use of placebos.

### Modified Release Formulations

In various aspects and embodiments, the modified release compositions in a solid oral dosage form comprise racemic amisulpride, or pharmaceutically acceptable salts thereof, and one or more pharmaceutically acceptable excipients.

It is to be understood that when an amisulpride enantiomer is said to be present in a certain weight amount, and such enantiomeric amisulpride is provided as a pharmaceutically acceptable salt thereof, that the weight amount refers to the amisulpride enantiomer portion exclusive of the salt portion, that is as the free base. Accordingly, it is to be understood that when a weight ratio of (R)-(+)-amisulpride to (S)-(−)-amisulpride is recited, it is the weight ratios only of the amisulpride portions exclusive of any salt portion especially if only one of the amisulpride enantiomers is present as a pharmaceutically acceptable salt thereof or the amisulpride enantiomers are present as different pharmaceutically acceptable salts.

In various aspects and embodiments, the modified release composition comprises a total amount of amisulpride between about 5 mg and about 1000 mg, between about 50 mg and about 750 mg, between about 5 mg and about 15 mg, between about 25 mg and about 50 mg, between about 50 mg and about 300 mg, or between about 100 mg and about 300 mg.

In various aspects and embodiments, the modified release compositions comprise a total amount of amisulpride from about 5 mg to about 15 mg, about 25 mg to about 50 mg, about 50 mg to about 100 mg, about 100 mg to about 1000 mg, from about 150 mg to about 800 mg, from about 100 mg to about 150 mg, from about 150 mg to about 200 mg, from about 200 mg to about 300 mg, from about 300 mg to about 400 mg, from about 400 mg to about 500 mg, from about 600 mg to about 700 mg, from about 700 mg to about 800 mg, about 100 mg, about 200 mg, about 300 mg. about 400 mg, about 500 mg, about 600 mg, about 700 mg, or about 800 mg, by weight of the free base.

It is to be understood that pharmaceutically acceptable excipients, include, but are not limited to, one or more binders, bulking agents, buffers, stabilizing agents, surfactants, wetting agents, lubricating agents, diluents, viscosity enhancing or reducing agents, emulsifiers, suspending agents, preservatives, antioxidants, opaquing agents, glidants, processing aids, colorants, sweeteners, taste-masking agents, perfuming agents, flavoring agents, diluents and other known additives to provide an elegant presentation of the drug or aid in the manufacturing of a medicament or pharmaceutical product comprising the modified release compositions described herein. Examples of carriers and excipients are described in detail in, e.g., Ansel, Howard C., et al., Ansel's Pharmaceutical Dosage Forms and Drug Delivery Systems. Philadelphia: Lippincott, Williams & Wilkins, 2004; Gennaro), Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005.

In various aspects and embodiments the modified release compositions comprise one or more pharmaceutically acceptable excipients, carriers, adjuvants, or vehicles, and are formulated as a solid oral dosage form. In various embodiments, the solid oral dosage form is in the form of a powder, tablet, caplet, or capsule. In various embodiments the solid oral dosage form comprises a tablet, and in various embodiments the solid oral dosage form comprises a capsule.

In various embodiments, the modified release compositions are formulated (for example, with respect to active ingredient amounts) to be administered once, twice, three times, or four times daily.

It is to be understood that the total amount of the amisulpride in the form of a racemic mixture of (R)-(+)-amisulpride and (S)-(−)-amisulpride, or pharmaceutically acceptable salts thereof, need not be provided in a single dosage unit form, e.g. a single tablet, capsule, etc. In various embodiments, the modified release composition is provided in dosage unit forms such that, for example, the administration of two of the dosage unit forms will result in administration of amisulpride in the desired combined amount of the (R)-amisulpride and (S)-amisulpride.

In various aspects and embodiments, all excipients comply with the respective The United States Pharmacopeia (USP), The Japanese Pharmacopoeia (JP), Japanese Pharmaceutical Excipients (JPE), The European Pharmacopoeia (Ph. Eur.), and/or The National Formulary (NF) monograph.

The modified release compositions are in various embodiments formulated in dosage unit form for ease of administration and uniformity of dosage. The expression “dosage unit form” as used herein refers to a physically discrete unit of agent appropriate for the subject to be treated.

### Tablet Formulations

In various embodiments, modified release compositions are provided as solid oral dosage forms in the form of a tablet comprising an intragranular component (granules) and an extragranular component; the intragranular component comprising (a) racemic amisulpride and (b) one or more pharmaceutically acceptable excipients; and the extragranular component comprising an extended release agent.

In various embodiments, the granules comprise between about 60% to about 80% by weight of racemic amisulpride, between about 10% to about 30% by weight of filler, between about 1% to about 5% by weight of binder; all weight percentages being exclusive of any solvent (e.g. water) removed during processing. In various embodiments, the resultant tablet (granules plus extragranular component) comprises between about 20% to about 70% by total tablet weight of granules, between about 10% to about 50% by total tablet weight of extended release agent, and a combined amount of both extragranular and intergranular filler that is between about 6% to about 60% by total tablet weight. In various embodiments, the combined amount of both extragranular and intergranular filler is between about 10% to about 50% by total tablet weight. In various embodiments, the resultant tablet comprises between about 20% to about 35% by total tablet weight of extended release agent.

In various embodiments, the granules comprise between about 60% to about 80% by weight of the total of racemic amisulpride, between about 10% to about 30% by weight of filler, between about 1% to about 5% by weight of binder; all weight percentages being exclusive of any solvent (e.g. water) removed during processing.

In various embodiments, the granules comprise between about 70% to about 80% by weight of racemic amisulpride, between about 20% to about 25% by weight of filler, between about 1° A) to about 5% by weight of binder; all weight percentages being exclusive of any solvent (e.g. water) removed during processing.

In still further embodiments, the granules comprise between about 75% by weight of weight of racemic amisulpride, about 22% by weight of filler, about 3% by weight of binder; all weight percentages being exclusive of any solvent (e.g. water) removed during processing.

In various embodiments, the resultant tablet (granules plus extragranular component) comprises between about 20% to about 70% by total tablet weight of granules, between about 10% to about 50% by total tablet weight of extended release agent, and a combined amount of both extragranular and intergranular filler that is between about 6% to about 60% by total tablet weight. In various embodiments, the resultant tablet comprises between about 20% to about 35% by total tablet weight of extended release agent.

In various embodiments, the combined amount of both extragranular and intergranular filler is between about 10% to about 50% by total tablet weight. In some embodiments, the resultant tablet (granules plus extragranular component) comprises between about 20% to about 70% by total tablet weight of granules, between about 10% to about 50% by total tablet weight of extended release agent, between about 0% to about 60% of extragranular filler, and between about 0% to about 2% of a lubricant by total tablet weight. In various embodiments, the resultant tablet comprises between about 20% to about 35% by total tablet weight of extended release agent.

In various embodiments, the resultant tablet (granules plus extragranular component) comprises between about 45% to about 65% by total tablet weight of granules, between about 10% to about 35% by total tablet weight of extended release agent, and between about 0% to about 40% total tablet weight of extragranular filler, and between about 0% to about 2% total tablet weight of a lubricant. In various embodiments, the resultant tablet comprises between about 20% to about 35% by total tablet weight of extended release agent.

In various aspects and embodiments, the ratio of the weight percentage of racemic amisulpride relative to the total combined weight percentage of the filler and binder in the granule is about 3:1.

In various aspects the ratio of the weight percentage of racemic amisulpride relative to the total combined weight percentage of the extragranular filler and extended release agent is about 1:1 to 1:0.8.

In various aspects and embodiments, granulated granules exhibit a D50 particle size of between about 180 microns to about 250 microns, between about 170 microns to about 190 microns, between about 175 microns to about 185 microns, between about 180 microns to about 205 microns, between about 205 microns to about 220 microns, or between about 220 microns to about 240 microns.

In various aspects and embodiments, blended granules plus extragranular component exhibit a D50 particle size of between about 180 microns to about 250 microns. between about 80 microns to about 120 microns, between about 90 microns to about 110 microns, between about 180 microns to about 205 microns, between about 205 microns to about 220 microns, or between about 220 microns to about 240 microns.

In still some further aspects and embodiments, the blended granules plus an extragranular component are compressed with a compression force of between 5-15kN to produce tablets having a hardness between about 70 N and about 170 N.

In various embodiments of 200 mg Matrix Tablet Formulations, (R)-amisulpride, (S)-amisulpride and D-mannitol are separately delumped with a screen mill. The delumped (R)-amisulpride, delumped (S)-amisulpride, delumped D-mannitol, and partly pregelatinized starch are granulated by spraying aqueous solution of partially hydrolyzed polyvinyl alcohol in a wet high-shear granulator, and wet granules are passed through a screening mill to give sized granules. D-mannitol and hypromellose are blended with the sized granules in a blender. Subsequently, magnesium stearate is blended with the granules in a blender. The blended granules are compressed into core tablets with a rotary press.

In various embodiments, examples of fillers include, but are not limited to, D-mannitol, dicalcium phosphate dibasic, microcrystalline cellulose, pregelatinized starch, and corn starch. It is to be understood that more than one type of filler can be used in a tablet of the present inventions and that the filler in the granules can be the same or different than that used in the extragranular component of the tablet.

In various embodiments, examples of binders include, but are not limited to, hydroxypropyl methylcellulose, partially hydrolyzed polyvinyl alcohol, polyvinyl alcohol, and methylcellulose.

In various embodiments, examples of extended release agents include, but are not limited to, hydroxpropylcellulose (HPC) and hypromellose (a.k.a. hydroxypropyl methylcellulose (HPMC)). It is to be understood that more than one type of extended release agent can be used in a tablet of the present inventions.

In various embodiments, examples of lubricants include, but are not limited to, magnesium stearate and sodium stearyl fumarate.

In various embodiments, the modified release tablets have a composition substantially in accord with that set forth in Table 1. The tablets of Table 1 each comprise 200 mg of racemic amisulpride, and varying amounts of extended release agent.

TABLE 1
Compositions Tablets 200 mg

| | | | Tab 1A | Tab 2A | Tab 3A |
|---|---|---|---|---|---|
| | | | (10%) | (15%) | (45%) |
| Component | | Function | mg/tab | mg/tab | mg/tab |
| Intra- | (R)-amisulpride | API | 100 | 100 | 100 |
| granular | (S)-amisulpride | API | 100 | 100 | 100 |
| component | D-Mannitol*¹ | Filler | 29.5 | 29.5 | 29.5 |
| | Pregelatinized starch | Filler | 29.5 | 29.5 | 29.5 |
| | Polyvinyl alcohol | Binder | 5.5 | 5.5 | 5.5 |
| | Purified water*² | Solvent | 72 | 72 | 72 |
| | (binder solvent) | | | | |
| Subtotal (granule component)*⁵ | | | 264.5 | 264.5 | 264.5 |
| Extra- | Hypromellose*³ | Extended | 50.0 | 75.0 | 225.5 |
| granular | | release | | | |
| component | | agent | | | |
| | D-Mannitol*⁴ | Filler | 178.0 | 153.0 | 2.5 |
| | Magnesium stearate | Lubricant | 7.5 | 7.5 | 7.5 |

| | | | |
|---|---|---|---|
| Total tablet weight | 500 | 500 | 500 |

*¹Crystalline powder, Pearlitol 50C (Roquette)
*²Water is removed during processing.
*³Metalose SR 90SH-100SR (Shin Etsu)
*⁴Spray dried powder, Pearlitol 100SD (Roquette)
*⁵After water removed during processing

In various embodiments, the modified release ablets having a composition substantially in accord with that set forth in Table 2. The tablets of Table 2 each comprise 200 mg of racemic amisulpride, and varying amounts of extended release agent.

TABLE 2
Compositions Matrix Tablets 200 mg

| | | | Tab 1B | Tab 2B | Tab 3B | Tab 4B | Tab 5B |
|---|---|---|---|---|---|---|---|
| | | | (10%) | (15%) | (25%) | (35%) | (45%) |
| Component | | Function | mg/tab | mg/tab | mg/tab | mg/tab | mg/tab |
| Intra- | (R)-amisulpride | API | 100 | 100 | 100 | 100 | 100 |
| granular | (S)-amisulpride | API | 100 | 100 | 100 | 100 | 100 |
| component | D-Mannitol^{*1} | Filler | 29.5 | 29.5 | 29.5 | 29.5 | 29.5 |
| | Partly pregelatinized | Filler | 29.5 | 29.5 | 29.5 | 29.5 | 29.5 |
| | starch | | | | | | |
| | Polyvinyl alcohol | Binder | 8 | 8 | 8 | 8 | 8 |
| | Purified water^{*2} (binder | Solvent | 72 | 72 | 72 | 72 | 72 |
| | solvent) | | | | | | |
| Subtotal (granule component)^{*5} | | | 267.0 | 267.0 | 267.0 | 267.0 | 267.0 |
| Extra- | Hypromellose^{*3} | Extended | 50.0 | 75.0 | 125.0 | 175.0 | 225.5 |
| granular | | release | | | | | |
| component | | agent | | | | | |
| | D-Mannitol^{*4} | Filler | 175.5 | 150.5 | 100.5 | 50.5 | — |
| | Magnesium stearate | Lubricant | 7.5 | 7.5 | 7.5 | 7.5 | 7.5 |

| | | | | | |
|---|---|---|---|---|---|
| Total tablet weight (mg) | 500 | 500 | 500 | 500 | 500 |

^{*1}Crystalline powder, Pearlitol 50C (Roquette)
^{*2}Water is removed during processing.
^{*3}Metolose SR 90SH-100SR (Shin Etsu)
^{*4}Spray dried powder, Pearlitol 100SD (Roquette)
^{*5}After water removed during processing

In various embodiments, the modified release tablets have a composition substantially in accord with that set forth in Table 3A. The tablets of Table 3A each comprise 200 mg of racemic amisulpride and varying amounts of extended release agent.

TABLE 3A
Compositions Matrix Tablets 200 mg

| | | | Tab 1C | Tab 2C | Tab 3C |
|---|---|---|---|---|---|
| | | | (10%) | (25%) | (15%) |
| Component | | Function | mg/tab | mg/tab | mg/tab |
| Intra- | (R)-amisulpride | API | 100 | 100 | 100 |
| granular | (S)-amisulpride | API | 100 | 100 | 100 |
| component | D-Mannitol*¹ | Filler | 29.5 | 29.5 | 29.5 |
| | Pregelatinized starch | Filler | 29.5 | 29.5 | 29.5 |
| | Polyvinyl alcohol | Binder | 5.5 | 5.5 | 5.5 |
| | Purified water*² | Solvent | 72 | 72 | 72 |
| | (binder solvent) | | 264.5 | 264.5 | 264.5 |
| Subtotal (granule component)*⁵ | | | | | |
| Extra- | Hypromellose*³ | Extended | 50.0 | 125.0 | 75.0 |
| granular | | release | | | |
| component | | agent | | | |
| | D-Mannitol*⁴ | Filler | 178.0 | 103.0 | 153.0 |
| | Magnesium stearate | Lubricant | 7.5 | 7.5 | 7.5 |

| | | | |
|---|---|---|---|
| Total tablet weight | 500 | 500 | 500 |

*¹Crystalline powder, Pearlitol 50C (Roquette)
*²Water is removed during processing.
*³Metalose SR 90SH-100SR (Shin Etsu)
*⁴Spray dried powder, Pearlitol 100SD (Roquette)
*⁵After water removed during processing

In various aspects and embodiments, the modified release composition, when tested using a two-stage in vitro dissolution test set forth in Table 4 and the accompanying description, (a) releases no more than about 40% of amisulpride after 2 hours and releases greater than about 80% of amisulpride in less than about 12 hours; (b) releases less than about 40% of amisulpride after 1 hour, releases more than about 20% and less than about 60% of amisulpride after 3 hours, and releases more than about 30% and less than 100% of amisulpride after 6 hours; (c) releases less than about 30% of amisulpride after 1 hour, releases more than about 20% and less than about 60% of amisulpride after 3 hours, and releases more than about 30% and less than about 75% of amisulpride after 6 hours; (d) releases less than about 20% of amisulpride after 1 hour, releases more than about 20% and less than about 50% of amisulpride after 3 hours_{;} and releases more than about 30% and less than about 75% of amisulpride after 6 hours; (e) releases more than about 30% and less than about 50% of amisulpride after 6 hours; (1) releases between about 30% and 75% of amisulpride after about 3 hours, and releases more than about 75% of amisulpride after about 12 hours; (g) releases less than about 30% of the amisulpride after 1 hour, releases more than 25% and less than about 55% of the amisulpride after 3 hours, and releases more than about 45% and less than about 75% of the amisulpride after 6 hours; or (h) releases more than about 75% of amisulpride after about 6 hours.

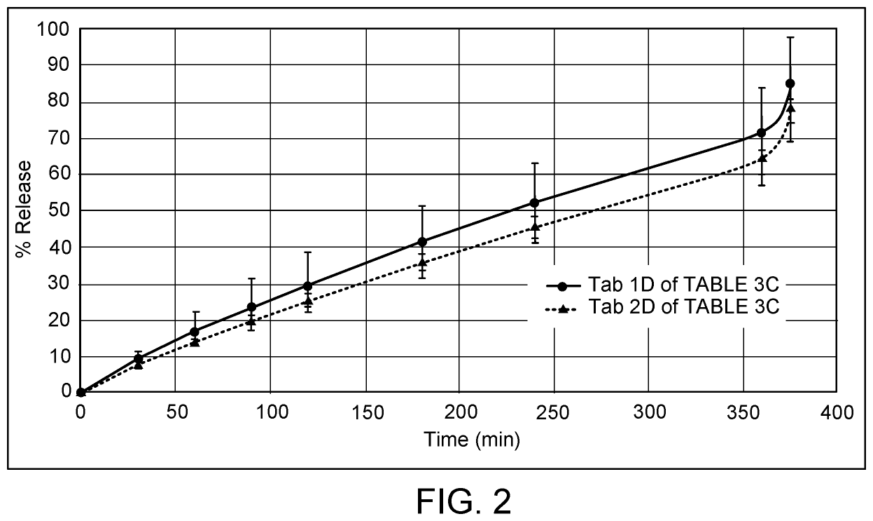

In various embodiments, the modified release composition has a release profile substantially in accord with that for Tab 1D (of Table 3C) in FIG. 2, or Tab 2D (of Table 3 C) in FIG. 2, when tested using a two-stage in vitro dissolution test set forth in Table 4 and the accompanying description.

TABLE 3B
Compositions Matrix Tablets 200 mg

| | | | Tab ID | Tab 2D |
|---|---|---|---|---|
| | | | (31.25%) | (25%) |
| | | | Oval, 11.2 × 8.7 mm | Round, 11 mm |
| Tablet shape, dimensions | | Function | mg/tab | mg/tab |
| Intra-granular | racemic-amisulpride | API | 200 | 200 |
| component | D-Mannitol *¹ | Filler | 29.5 | 29.5 |
| | Pregelatinized starch | Filler | 29.5 | 29.5 |
| | Polyvinyl alcohol *⁶ | Binder | 8 | 8 |
| | Purified water*² | Solvent | q.s. | q.s. |
| Subtotal (granule component) *⁵ | | | 267 | 267 |
| Extra-granular | Hypromellose *³ | Extended | 125 | 125 |
| component | D-Mannitol *⁴ | release agent | — | 98 |
| | Aerosil 200 | Filler | 2 | 2.5 |
| | Magnesium stearate | Glidant | 6 | 7.5 |
| | | Lubricant | | |

| | | |
|---|---|---|
| Total tablet weight (mg) | 400 | 500 |

*¹: Crystalline powder, Pearlitol 50C (Roquette)
*²: Water is removed during processing,
*³: Metolose SR 90SH-100SR (Shin Etsu) (viscosity: l00mPa s)
*⁴: Spray dried powder, Pearlitol 100SD (Roquette)
*⁵: After water removed during processing
*⁶: Dissolved in purified water,
q.s. means quantum sufficiat (as much as necessary)

TABLE 3C
Data of FIG. 2 (data is % API released vs Time)

| | Tab 1D | Tab 2D |
|---|---|---|
| Time | (31.25%) | (25%) |
| (hours) | 200 mg | 200 mg |
| 0 | 0 | 0 |
| 0.5 | 9.0 | 7.6 |
| 1 | 16.8 | 14.0 |
| 1.5 | 23.6 | 19.7 |
| 2 | 29.8 | 25.2 |
| 3 | 41.4 | 35.7 |
| 4 | 52.2 | 45.7 |
| 6 | 71.6 | 64.5 |
| 6.25 | 85.0 | 78.3 |

TABLE 4
In-vitro Dissolution Test Parameters MR Tablet Formulations

| | |
|---|---|
| Medium: | 0-60 minutes 500 mL 0.01 M HC1, pH 2.0 |
| | For pH Switch |
| | Add 400 mL of 0.15M Na3PO4 (pre-heat to 37° C.), pH 6.8 ± 0.05 |
| Dissolution type: | USP II (Paddles) |
| Paddle Speed: | 75 rpm |
| Volume of Medium: | 0-60 minutes 500 mL |
| | 60 minutes onwards 900 mL |
| Temperature: | 37.0° C. (± 0.5) |
| Sampling time points: | Stage 1: 0.5, 1 |
| | Stage 2: 1.5, 2, 3, 4, 6, 8,10, 12 hours followed by infinity for 1 hour |
| | @ 250 rpm |
| Sampling Type: | Automatic with filter 10 μm full flow |
| Sampling Volume: | 1.5 mL |

The in vitro dissolution profiles of the modified release (MR) formulations of Table 3B in FIG. 2 were acquired using a paddle apparatus substantially in accord with that described by the United States Pharmacopeia Convention (USP) Apparatus 2 of Chapter 711 Dissolution; USP41-NF36 General Chapter <711> Dissolution. The apparatuses were operated as described in Table 4. Amisulpride release was determined from 1.5 ml samples taken from the medium and analyzed using HPLC with a Kinetex Biphenyl, 4.6×100 mm, 2.6 μm (P/N: 00D-4622-E0) column and UV detector set to 280 nm at the time points indicated in the figure.

A variety of procedures can be used to make the modified release tablets described herein. For example, the modified release tablets of Tables 1-3A and 3B, can be made as follows. The active pharmaceutical ingredients ((R)-amisulpride and (S)-amisulpride) and D-mannitol (Pearitol 50C) are separately delumped with a screen mill. The delumped API, delumped D-mannitol and partly pregelatinized starch are granulated by spraying aqueous solution of partially hydrolyzed polyvinyl alcohol in a wet high-shear granulator, and wet granules are passed through a screening mill and dried in a fluid bed granulator. The resultant granules can then passed through a screening mill to give sized granules. D-mannitol (Pearitol 100SD) and hypromellose are then blended with the sized granules in a blender. Subsequently, magnesium stearate is blended with the granules in a blender. The blended granules are then compressed into core tablets with a rotary press.

More specifically, prior to mixing the active pharmaceutical ingredients ((R)-amisulpride and (S)-amisulpride) with the various excipients to form granules, the (R)-amisulpride and (S)-amisulpride are separately delumped. The delumping can employ a Powrex(Quadro) Co mill QC-194S, configured with a round bar impeller and round holed screen having screen size 1.397 mm (055R), with a spacer size of 0.200, and the impeller operated with a low rotating speed of 743 min⁻¹. D-Mannitol can also be delumped by a similar procedure.

Granulation can be achieved using a Powrex FM-VG-05 (total capacity: 5 L) Granulator, configured with a blade of straight type 35° (rotating at 400 rpm), cross screws of 60 mm×3 plates, (rotating at 3000 rpm), seal air pressures of 30 NL/min (Blade), 20 NL/min (Cross screw), a two fluid nozzle spray gun (with spray gun nozzle size of 1.0 mm operated at a spray rate of 10 g/min, a spray air pressure of 0.03 MPa, and a temperature control jacket set as required for various steps in the process.

The binder is first prepared as a 10% solid concentration placed in purified water is heated above 80° C. and partially hydrolyzed polyvinyl alcohol is dissolved in the heated water by propeller mixer. In addition, as required, other excipients can be delumped prior to combination.

To produce granules and tablets substantially in accord with those of Tables 1-3A and 3B, the binder is added for introduction via the spray guns, and is delumped mannitol, partly pregelatinized starch, delumped API (e.g delumped racemic amisulpride, or delumped (R)-amisulpride and delumped (S)-amisulpride) are mixed briefly in a plastic bag. The resultant mixture is added to the granulator container and blended for 1 min., then the sprayer started to start spraying the binder. After spraying, all granules in the container, including granules adhered on surface of container, blade, cross screw, lid, are scraped off and the loss of water on drying is determined.

The resultant granules are then wet sized prior to combination with the extra-granular component. The granules can be wet sized using a Powrex(Quadro) Co mill QC-194S, configured with a round bar impeller and round holed screen having screen size: 3.962 mm(156R), with a spacer size of 0.225, and the impeller operated with a low rotating speed of 900 min⁻¹. The granules are fed manually over 2-3min (for a 300 g scale feed).

The wet sized granules are then dried using a Powrex FD-MP-01(total capacity: 0.6-3 L), with an inlet air flow of 0.7-1.0 m³/hr having an inlet air temperature of 80° C. The wet sized granules are added to the container and drying started. The drying is stopped when the outlet air temperature reached 40° C., and the granules are tested for loss of water; loss on drying (LOD) should be NMT 2.0%.

The granules and extra-granule components can be blended using a Tsutsui scientific instrument S-3 (V-blender, total capacity: 2 L) as follows. The sized granules are added to the container of the blender, then the extended release agent (e.g. hypromellose) and filler (e.g., D-mannitol) are added, and the material blended for 15 min at 40 rpm. A portion of the blended granules are removed and mixed with a lubricant (e.g., magnesium stearate), the mixture is passed through an appropriate sieve (e.g., a 850 μm sieve), and the sieved mixture is added back to the blender container, and blended for 5 min at 40 rpm.

The tablets of Tables 1-3A and 3B can be formed using a Rotary press Kikusui VEL2, with 11 WR (22.0R, 5.5 R) tooling, operated at a compression speed of 20 rpm and the compression force adjusted to produce tablets having a hardness of about NLT 100N.

### Multiparticulate Capsule (MUPS) Formulations

In various aspects and embodiments, modified release compositions are provided as solid oral dosage forms in the form of a capsule comprising multiple coated particulates; the particulate component comprising (a) coated particulates of substantially enantiomerically pure (R)-amisulpride and (b) coated particulates of substantially enantiomerically pure (S)-amisulpride, where R and S amisulpride particulates are combined in the capsule in a ratio of R:S amisulpride of about 50:50 by weight of free base.

In various embodiments, the coating of the (R)-amisulpride and (S)-amisulpride particles is substantially the same, and in various embodiments, the coating of the (R)-amisulpride and (S)-amisulpride particles differs. In various embodiments, the particulates are coated with one or more polymer coatings comprising between about 8% and about 60%, between about 10% and about 45%, or between about 15% and about 30% by weight of the total particle weight.

It is to be understood that the weight percentage of the polymer coat can also be described as the weight of the polymer coating (e.g. polymer +plasticizer) as a percentage of the weight of the uncoated particles. Accordingly, in various embodiments modified release compositions are provided as solid oral dosage forms in the form of a capsule comprising multiple coated particulates; the particulate component comprising (a) coated particulates of substantially enantiomerically pure (R)-amisulpride and (b) coated particulates of substantially enantiomerically pure (S)-amisulpride, R and S amisulpride particulates combined in the capsule in a ratio of R:S amisulpride of about 50:50 by weight of amisulpride free base. In various embodiments, the coating of the (R)-amisulpride and (S)-amisulpride is substantially the same, and in various embodiments, the coating of the (R)-amisulpride and (S)-amisulpride particles differs. In various embodiments, the particulates are coated with one or more polymer coatings comprising between about 10% and about 60%, between about 10% and about 45%, or between about 15% and about 35% by weight of the uncoated particle weight.

In various embodiments, the particulates comprise in addition to the API, a binder and optionally a lubricant excipient, the combined API, binder and lubricant particulates being coated with one or more polymers. In various embodiments, the API comprises between about 35% and about 65% of the total coated particle weight, the binder comprises between about 8% and about 20%, and in various embodiments between about 9% and about 15%, of the total coated particle weight, the lubricant excipient comprises between about 8% and about 20%, and in various embodiments between about 9% and about 15%, of the total coated particle weight, and the polymer coating between about 10% and about 45% by weight of the total particle weight.

In various embodiments, the particulates comprise in addition to the API, a binder and optionally a lubricant excipient, and the combined API, binder and lubricant particulates being coated with one or more polymers. In various embodiments, the API comprises between about 40% and about 85% of the total uncoated particle weight (and in various embodiments between about 65% and about 75% of the total uncoated particle weight), the binder comprises between about 8% and about 20%, and in various embodiments between about 9% and about 15%, of the total uncoated particle weight, the lubricant excipient comprises between about 8% and about 20%, and in various embodiments between about 9% and about 15%, of the total uncoated particle weight, and the polymer coating between about 10% and about 60% by weight of the uncoated particle weight, and in various embodiments between about 10% and about 45% by weight of the uncoated particle weight, and in various embodiments between about 15% and about 35% by weight of the uncoated particle weight.

In various embodiments, the ratio of the API to polymer coating is between about 1:0.5 and 1:0.6. In various embodiments, the ratio of the API to binder is between about 1:0.2 and 1:0.25. In various further embodiments, the ratio of the API to polymer coating is between about 1:0.5 and 1:0.6, and the ratio of the API to binder is between about 1:0.2 and 1:0.25.

In various embodiments, examples of binders include, but are not limited to, hydroxypropyl cellulose, hydroxypropyl methylcellulose, partially hydrolyzed polyvinyl alcohol, polyvinyl alcohol, methylcellulose, hydroxyethylcellulose, hydroxymethylcellulose, carboxymethylcellulose, polyvinylpyrrolidone, copolyvidone, polyethylene glycol, polyvinyl alcohol-acrylic acid-methyl methacrylate copolymer, vinyl acetate-vinylpyrrolidone copolymer, polyvinyl alcohol-polyethylene glycol-graft copolymer, pregelatinized starch, dextrin, dextran, pullulan, alginic acid, gelatin, pectin, and a mixture of one or more thereof. In various embodiments, one or more of hydroxypropyl cellulose and polyvinyl alcohol are used.

In various embodiments, examples of lubricant excipients include, but are not limited to, micronized talc, magnesium stearate, sodium stearyl fumarate, hydrated silicon dioxide, magnesium silicate, light anhydrous silicic acid, synthetic aluminum silicate, heavy anhydrous silicic acid, silicon dioxide, calcium stearate, aluminum stearate, potassium stearate, zinc stearate, yellow ferric oxide, red ferric oxide, and titanium oxide. In various embodiments, one or more of talc, magnesium stearate and sodium stearyl fumarate are used.

In various embodiments, the polymer coating comprises one or more water insoluble polymers and one or more plasticizers mixed with the one or more polymers. In various embodiments, examples of water insoluble polymers include, but are not limited to, ethylcellulose, acetyl cellulose, aminoalkylmethacrylate copolymer RS, ethyl acrylate, and vinyl acetate resin. In various embodiments, examples of plasticizers include, but are not limited to, triethyl citrate, polyethylene glycol, propylene glycol, polypropylene glycol, sorbitol sorbitan solution, triacetin, glycerin, glycerol fatty acid, silicon oil, acetyltriethyl citrate, diethyl phthalate, tributyl citrate, dibutyl phthalate, acetyltributyl citrate, dibutyl sebacate, glycerol triacetate, and acetylated monoglyceride. In various embodiments, one or more of ethylcellulose and aminoalkylmethacrylate copolymer RS are the polymers, and triethyl citrate is the plasticizer. In various embodiments, the polymer coating comprises a mixture of ethylcellulose and triethyl citrate where the weight ratio of ethylcellulose to triethyl citrate is in the range between about 3:1 to about 5:1 and in various embodiments about 4:1.

It is to be understood that in the present multiparticulate capsule formulations the R-amisulpride and the S-amisulpride containing particulates may be formulated and coated separately, and then sufficient portions of the R-amisulpride particulates and the S-amisulpride particulates are combined in a capsule to provide the desired racemic amisulpride. It is to be understood that if the percentage of amisulpride in a coated particulate differs between the R-amisulpride particulates and the S-amisulpride particulates (as may result from different uncoated particulate formulations and/or different amounts of polymer coating), then particulates are combined based on the weight of the amisulpride in the respective particulates.

Accordingly, it is to be understood that the absolute weights of the capsule formulations in Tables 10 and 11 are not indicative of the absolute weights of the various components in a final multiparticulate capsule comprising the desired racemic amisulpride R:S ratio. However, the compositions of Tables 10 and 11 do provide the relative ratios of the various components in the respective particulates, R-amisulpride particulates in Table 5 and the S-amisulpride particulates in Table 6, of the particulate components of a multiparticulate capsule in various embodiments.

In various embodiments, the modified release multiparticulate capsules have an R-amisulpride particulate relative component composition substantially in accord with that set forth in Table 5; that is, the weight ratios of the various components in the R-amisulpride particulates in the multiparticulate capsules are substantially in accord with the ratios (not absolute weights) set forth in Table 5.

In various embodiments, the present inventions provide modified release pharmaceutical multiparticulate capsules having an S-amisulpride particulate relative component composition substantially in accord with that set forth in Table 6; that is, the weight ratios of the various components in the S-amisulpride particulates in the multiparticulate capsules are substantially in accord with the ratios (not absolute weights) set forth in Table 6.

TABLE 5
Compositions R-Amisulpride Particulate Formulations

| | | Quantity (mg) | | |
|---|---|---|---|---|
| | | | Lot RC10 | Lot RC40 |
| Component | Function | IR particles | (10%) | (40%) |
| (R)-amisulpride | API | 200 | 200 | 200 |
| Hydroxypropyl | Binder | 44.0 | 44.0 | 44.0 |
| Cellulose | | | | |
| Micronized Talc | Lubricant | 44.4 | 44.4 | 44.4 |
| Ethylcellulose | Polymer in | — | 23.3 | 93.0 |
| | Polymer Coat | | | |
| Triethyl Citrate | Plasticizer in | — | 5.6 | 22.3 |
| | Polymer Coat | | | |
| Total Weight | | 288.4 | 317.2 | 403.8 |

TABLE 6
Compositions S-Amisulpride Particulate Formulations

| | | Quantity (mg) | | | | | | |
|---|---|---|---|---|---|---|---|---|
| | | IR | Lot SC10 | Lot SC20 | Lot SC30 | Lot SC40 | Lot SC50 | Lot SC60 |
| Component | Function | particles | (10%) | (20%) | (30%) | (40%) | (50%) | (60%) |
| (S)-amisulpride | API | 200 | 200 | 200 | 200 | 200 | 200 | 200 |
| Hydroxypropyl | Binder | 44.0 | 44.0 | 44.0 | 44.0 | 44.0 | 44.0 | 44.0 |
| Cellulose | | | | | | | | |
| Micronized | Lubricant | 44.6 | 44.6 | 44.6 | 44.6 | 44.6 | 44.6 | 44.6 |
| Talc | | | | | | | | |
| Ethylcellulose | Polymer in | — | 23.3 | 46.5 | 69.8 | 93.1 | 116.4 | 139.6 |
| | Polymer | | | | | | | |
| | Coat | | | | | | | |
| Triethyl Citrate | Plasticizer | — | 5.6 | 11.2 | 16.8 | 22.3 | 27.9 | 33.5 |
| | in | | | | | | | |
| | Polymer | | | | | | | |
| | Coat | | | | | | | |
| Total Weight | | 288.6 | 317.5 | 346.3 | 375.2 | 404.04 | 432.9 | 461.8 |

The particle size distributions for the particulates comprising the formulations of Tables 10 and 11 are shown in Tables 12 and 13, respectively. These particle size distributions were determined using a sieve analysis; the particulates being shifted through a stack of wire mesh sieves (conforming to BS 410 and ISP 3310-1 standards and with nominal aperture opening sizes as indicated in columns 1 of Tables 12 and 13) that are shaken to separate the particulates into discrete size ranges.

TABLE 7
Particle Size Distribution (PSD) for Particulates in Formulations of Table 5
Fraction Percentage by Sieve Size

| | | Lot RC40 |
|---|---|---|
| Sieve size Microns | IR Particles | (40%) |
| >500 | 0.25 | 0.0 |
| >355 | 0.49 | 1.7 |
| >250 | 0.82 | 22.0 |
| >212 | 24.96 | 35.0 |
| >180 | 33.96 | 17.4 |
| >125 | 27.33 | 21.3 |
| >90 | 2.86 | 2.4 |
| >63 | 1.64 | 0.3 |
| Base/<75 | 0.49 | 0.0 |

TABLE 8
Particle Size Distribution (PSD) for Particulates in Formulations of Table 6
Fraction Percentage by Sieve Size

| | | Lot SC30 | Lot SC60 |
|---|---|---|---|
| Sieve size Microns | IR Particles | (30%) | (60%) |
| >500 | 0.00 | 0.00 | 0.24 |
| >355 | 1.49 | 1.98 | 1.72 |
| >250 | 1.75 | 11.13 | 24.08 |
| >212 | 20.45 | 40.60 | 50.61 |
| >180 | 43.14 | 36.88 | 17.94 |
| >125 | 28.93 | 9.16 | 5.16 |
| >90 | 1.24 | 0.25 | 0.25 |
| >63 | 0.25 | 0.00 | 0.00 |
| Pass | 2.75 | 0.00 | 0.00 |
| D50 (gm) | 192.40 | 215.40 | 232.00 |

In various embodiments, the modified release multiparticulate capsules have a composition substantially in accord with that set forth in Table 9. The tablets of Table 9 each comprise 200 mg or 100 mg of (R)-amisulpride and (S)-amisulpride in the ratio R:S of 50:50, and varying amounts of polymer coating for the particulates. As described herein, the multiparticulate capsules are produced by combining appropriate amounts of polymer coated (R)-amisulpride particulates and polymer coated (S)-amisulpride particulates within a capsule.

TABLE 9
Compositions Multiparticulate
Capsules

| | | Cap | Cap | Cap | Cap |
|---|---|---|---|---|---|
| | | C1A | C1B | C1C | C1D |
| | | (10%) | (40%) | (40%) | (10%) |
| Component | Function | mg | mg | mg | mg |
| (R)-amisulpride | API | 100 | 100 | 50 | 50 |
| (S)-amisulpride | API | 100 | 100 | 50 | 50 |
| Hydroxypropyl | Binder | 44.0 | 44.0 | 22.0 | 22.0 |
| Cellulose | | | | | |
| Micronised Talc | Lubricant | 44.4 | 44.4 | 22.2 | 22.2 |
| Ethylcellulose | Polymer Coat | 23.2 | 93.0 | 46.5 | 11.6 |
| Triethyl Citrate | Polymer Coat | 5.5 | 22.3 | 11.2 | 2.8 |
| Total per Capsule | | 317.2 | 403.8 | 201.9 | 158.6 |
| Gelatin Capsules | Encapsulation | 1 unit | 1 unit | 1 unit | 1 unit |

A variety of procedures can be used to make the modified release capsules described herein. For example, the (R)-amisulpride particulates of Table 5, the (S)-amisulpride particulates of Table 6, and the coated particulates of substantially enantiomerically pure (R)-amisulpride and coated particulates of substantially enantiomerically pure (S)-amisulpride used to make the modified release capsules of Table 9, were made as follows. The uncoated substantially enantiomerically pure (R)-amisulpride particulates and uncoated substantially enantiomerically pure (S)-amisulpride particulates were made separately using the same procedure; and coated separately to make modified release particulates using the same procedure.

The uncoated particulates were made by as follows. The active pharmaceutical ingredients ((R)-amisulpride and (S)-amisulpride) were separately delumped with a screen mill and the binder (hydroxypropyl cellulose) was separately delumped with a sieving shaker. The delumped active pharmaceutical particulates ingredients were each separately combined with the delumped hydroxypropyl cellulose and micronized talc (lubricant), blended and then granulated by spraying purified water in a wet high-shear granulator to make wet particulates, and wet particulates were then dried in a fluid bed granulator. The resultant dry particulates were sieved with sieving shaker to obtain the immediate release (IR) particulates. The resultant dry IR particulates were then coated (each enantiomer separately) to make the modified release (MR) particulates for each enantiomer.

More specifically, prior to mixing the active pharmaceutical ingredients ((R)-amisulpride and (S)-amisulpride) with the various excipients to form granules, the (R)-amisulpride and (S)-amisulpride are separately delumped. The delumping employed a Powrex(Quadro) Co mill QC-194S, configured with a round bar impeller and round holed screen having screen size 1.143 mm, and the impeller operated with a low rotating speed of 743 min⁻¹. The hydroxypropyl cellulose delumping employed an IIDA Sieving shaker (ES-65), with a 150 mmϕ sieve, and sieve mesh sizes of 150 μm and 500 μm, with the shaking level rotating at 230 rpm and tapping at 130 rpm, and a total sieving time of 10 minutes.

Granulation was achieved using a Powrex FM-VG-05 (total capacity: 5 L) Granulator, configured with a blade of straight type 35° (rotating at 400 rpm), cross screws of 60 mm×3 plates, (rotating at 3000 rpm), seal air pressures of 20 NL/min (Blade), 10 NL/min (Cross screw), a two fluid nozzle spray gun (with spray gun nozzle size of 0.5 mm ID, length of nozzle tip to air cap of 0.5 mm and operated at a spray rate of 4 g/min, a spray air pressure of 0.08 MPa. It is to be understood that a temperature control jacket can be used and set as required for various steps in the process.

The procedure for granulation was as follows. The talc was added to the granulator container and blended for 1 minute. The sieved hydroxypropyl cellulose in propoer proportion was added to the delumped API ((R)-amisulpride or (S)-amisulpride) in a plastic bag and mixed shortly. The resultant mixture was added to the granulator container (containing talc) and blended for 3 minutes. The spray binder (purified water) was then started and sprayed in the following amounts and blended in the following eleven aliquots: aliquot 1 sprayed 50 g; aliquot 2 sprayed 50 g; aliquot 3 sprayed 25 g; aliquot 4 sprayed 0 g (blended for 5 min); aliquot 5 sprayed 15 galiquot 6 sprayed 0 g (blended for 5 min); aliquot 7 sprayed 15 g; aliquot 8 sprayed 0 g (blended for 5 min); aliquot 9 sprayed 0 g (blended for 3 min); aliquot 10 sprayed 0 g (blended for 2 min); and aliquot 11 sprayed 0 g (blended for 2 min). After spraying, all granules in container including granules adhered on surface of container, blade, cross screw, and lid, were scraped off and the steps of spraying and blending for the 11 aliquots and scraping were repeated. As mentioned previously, it is to be understood that this process was carried out separately for each of the APIs ((R)-amisulpride and (S)-amisulpride), that is a given batch contained substantially only a single amisulpride enantiomer.

After granulation, the resulting particulates were dried using a Powrex FD-MP-01 (Total capacity: 0.6-3 L) operated with an inlet air flow of 0.79-0.91 m³/min, and an nlet air temperature of 80° C. The wet particulates were added to the container and drying started. The drying was stopped when the outlet air temperature reached 40° C., and the particulates tested for loss of water; loss on drying (LOD) should be NMT 2.0%.

The dried particulates were then sieved (separately for each enantiomer) using an IIDA Sieving shaker (ES-65), with a 150 mmϕ sieve, and sieve mesh sizes of 106 μm and 500 μm, with the shaking level rotating at 230 rpm and tapping at 130 rpm, and a total sieving time of 10 minutes. The resultant immediate release particulates were then coated to prepare the MR particulates.

Specifically, IR particulates of a single enantiomer were coated with a Powrex/FD-MP-01/SPC (Total capacity: 0.6-3 L) gas suspension/fluidized bed apparatus in 650 g batches configured with an inlet air flow of 0.77-0.94 m³/min, a SPC pulse air pressure of 0.2 MPa, a two fluid nozzle spray gun (with spray gun nozzle size of 1.2 mm ID, length of nozzle tip to air cap of 2.0 mm and operated at a spray rate of 4 g/min, a spray air pressure of 0.2 MPa), and with preheating to provide an initial inlet temperature of 67° C. and outlet target temperature of 38° C., that exhibited a range of 36-40° C. After preheating, the IR particulates of a specific enantiomer were added to the container and granulation and spraying started. Coating amount was monitored (if necessary) by weight and the granulator was stopped when the sprayed amount reached the desired coating level. As mentioned previously, it is to understood that this process was carried out separately for each of the APIs ((R)-amisulpride and (S)-amisulpride), that is a given batch contained substantially only a single amisulpride enantiomer.

The coated particulates were then dried using a TABAI/perfect oven PH-400 (a direct heating, static solid bed drier with tray and trucks) operated at 60° C., with metallic tray and a 1.5 cm thick particulate layer on the tray, and dried for 18 hours. Subsequent to drying, the particulates were sieved with a 500 μm sieve by hand.

### (R)-(+)-amisulpride and (S)-(−)-amisulpride

The modified release formulations comprise racemic amisulpride, or pharmaceutically acceptable salts thereof.

In various embodiments, the total combined amount of (R)-amisulpride and (S)-amisulpride in is about 5 mg, 10 mg, 20 mg, 25 mg, 50 mg, 75 mg, 100 mg, 150 mg, 200 mg, 300 mg, 400 mg, 500 mg, 600 mg, 700 mg, 750 mg, 800 mg, 900 mg, or 1000 mg. In various embodiments, once administered, or as administered over a treatment cycle, the total combined amount of (R)-(+)-amisulpride and (S)-(−)-amisulpride ranges from about 50-1000 mg, from about 200-750 mg, from about 50-100 mg, or about 5-15 mg.

In various embodiments, the combined amount of (R)-amisulpride and (S)-amisulpride is about: 5 mg, 10 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg. 650 mg, 675 mg, 700 mg, 725 mg, 750 mg, 775 mg, 800 mg, 825 mg, 850 mg, 875 mg, 900 mg, 925 mg, 950 mg, 975 mg, or 1000 mg, by weight of the free base.

### Methods of Treatment

The medicaments and modified release compositions can be used to treat, and/or used to manufacture a medicament to treat, a psychiatric disorder in a subject, a neurological disorder in a subject, or both a neurological disorder and a psychiatric disorder, the disorder includingpersistent depressive disorder (PDD), schizophrenia, acute schizophrenia; chronic schizophrenia; positive symptoms of schizophrenia (e.g. delusions, hallucinations, thought disorders); and negative symptoms of schizophrenia.

In various aspects, the medicaments and modified release compositions can be used to treat, and/or used to manufacture a medicament to treat and/or manage postoperative nausea and vomiting.

In various aspects and embodiments, there is provided a method of treating a psychiatric disorder in a subject comprising administering to the subject a modified release composition in a solid oral dosage form comprising racemic amisulpride, or pharmaceutically acceptable salts thereof, and one or more pharmaceutically acceptable excipients.

In various embodiments of the method of treating:

(1) the modified release composition when administered to a subject population results in a population average maximum QT interval prolongation of (a) less than about 0.45 milliseconds (ms) per 10 mg of amisulpride for the time period of 12 hours after administration; (b) less than about 0.30 milliseconds (ms) per 10 mg of amisulpride; (c) less than about 0.20 milliseconds (ms) per 10 mg of amisulpride; (d) less than about 0.15 milliseconds (ms) per 10 mg of amisulpride; (e) less than about 0.10 milliseconds (ms) per 10 mg of amisulpride t; (f) less than about 0.05 milliseconds (ms) per 10 mg of amisulpride; or (g) less than about 0.02 milliseconds (ms) per 10 mg of amisulpride; and/or

(2) the solid oral dosage form, when dissolution tested using a two-stage in vitro gastrointestinal simulation dissolution test, e.g. such as described in Table 5 and accompanying text, releases (a), less than about 40% of amisulpride after 1 hour, more than about 20% and less than about 60% of amisulpride agent after 3 hours, and more than about 30% and less than 100% of amisulpride after 6 hours; (b) less than about 30% of amisulpride after 1 hour, more than about 20% and less than about 60% of amisulpride after 3 hours, and more than about 30% and less than about 75% of amisulpride after 6 hours; (c) less than about 20% of amisulpride after 1 hour, more than about 20% and less than about 50% of amisulpride after 3 hours, and more than about 30% and less than about 75% of amisulpride after 6 hours; (d) more than about 30% and less than about 50% of amisulpride after 6 hours; (e) between about 30% and 75% of amisulpride after about 3 hours and more than about 75% of amisulpride after about 12 hours; (0 releases less than about 30% of the amisulpride after 1 hour, releases more than 25% and less than about 55% of the amisulpride after 3 hours, and releases more than about 45% and less than about 75% of the amisulpride after 6 hours; or (g) more than about 75% of amisulpride after about 6 hours; and/or

(3) the modified release composition, when administered to a subject population, is effective in minimizing fluctuations between Ginn and Cmax of amisulpride; and/or

(4) the modified release composition used in treating the psychiatric disorder is effective in minimizing the difference between Cmin and Cmax of amisulpride compared to an immediate release composition having the same total daily amount of amisulpride as the modified release pharmaceutical composition, wherein the value of Cmin is that at about 9 hours after administration; and/or

(5) the modified release composition, when administered to a subject population, is effective in providing a ratio of Cmax/Cmin of amisulpride that is less than about 2, less than about 1.9, or less than about 1.8, wherein the value of Cmin is that at about 9 hours after administration; and/or

(6) when the modifiedrelease composition is administered to a subject population (i) the area under the curve (AUC) of blood plasma concentration versus time of amisulpride from administration to Tmax (AUCo-Tmax) is less than about 17%, less than about 16%, less than about 14%, or less than about 12% of the area under the curve from administration to 48 hours (AUC₀₋₄₈); and (ii) Tmax of amisulpride is between about 4 and about 6 hours after administration; and/or

(7) the modified release composition when administered to a subject population provides (i) a blood plasma Cmax of amisulpride that is less than about 75%, 70%, 65%, 60%, 55%, or 50% of the Cmax achieved by an immediate release formulation having the same total daily amount of amisulpride as in the modified release composition.

In various embodiments, the disorder is one or more of persistent depressive disorder (PDD), schizophrenia, acute schizophrenia; chronic schizophrenia; positive symptoms of schizophrenia (e.g. delusions, hallucinations, thought disorders);and negative symptoms of schizophrenia.

### Treatment Cycle

It is to be understood that the administration of an amount of amisulpride over a treatment cycle may be provided in a multiple dosage regimen. For example, in various embodiments, a multiple dosage regimen comprises dosage with two or more modified release dosage unit forms substantially simultaneously; dosage with two or more modified release dosage unit forms sequentially; dosage with two or more modified release dosage unit forms within a period of time from one another, in various embodiments within 4 to 48 hours from one another; and combinations thereof

In various embodiments, the treatment cycle is daily and the administration occurs: (a) once per day; (b) twice per day; (c) thrice per day; or (d) four times per day. In various embodiments, the treatment cycle is every two days.

In various embodiments, the treatment cycle is daily and the total amount of racemic amisulpride is about 200 mg over the treatment cycle. In various embodiments, the treatment cycle is daily and the total amount of (racemic amisulpride is about 400 mg over the treatment cycle.

### Disorders

The Diagnostic and Statistical Manual of Mental Disorders, Fifth Ed., hereinafter, the “DSM-5”), published by the American Psychiatric Association in 2013, and is incorporated herein by reference, provides a standard diagnostic system upon which persons of skill rely for diagnosis of various diseases and disorders.

In various aspects, the disease or disorder which the medicaments and methods treat comprises one or more of a psychiatric disorder; such as schizophrenia; acute schizophrenia; chronic schizophrenia; positive symptoms of schizophrenia (e.g. delusions, hallucinations, thought disorders); negative symptoms of schizophrenia; persistent depressive disorder (PDD); anxiety disorder; obsessive-compulsive disorder; behavior disturbances associated with a neurocognitive disorder; conduct disorder; neurological disorder; medication-induced movement disorder; and motor disorder. In various aspects, a condition which the medicaments and methods treat comprises postoperative nausea and vomiting.

In various embodiments, the neurological or psychiatric disease or disorder is one or more of, schizophrenia, acute schizophrenia; chronic schizophrenia; positive symptoms of schizophrenia (e.g. delusions, hallucinations, thought disorders); negative symptoms of schizophrenia, persistent depressive disorder (PDD); and treatment resistant depression (TRD).

In various embodiments, the neurological or psychiatric disease or disorder is selected from a psychosis, including schizophrenia (paranoid, disorganized, catatonic or undifferentiated), schizophreniform disorder, delusional disorder, brief psychotic disorder, shared psychotic disorder, psychotic disorder due to a general medical condition and substance-induced or drug-induced (e.g., phencyclidine, ketamine and other dissociative anesthetics, amphetamine and other psychostimulants and cocaine) psychotic disorder, psychosis disorder, psychosis associated with affective disorders, brief reactive psychosis, schizoaffective psychosis, “schizophrenia-spectrum” disorders such as schizoid or schizotypal personality disorders, or illnesses with associated psychosis (such as major depression, Alzheimer's disease and post-traumatic stress syndrome), including both positive, negative, and cognitive symptoms of schizophrenia and other psychoses; anxiety disorders including acute stress disorder, agoraphobia, generalized anxiety disorder, obsessive-compulsive disorder and related disorders including body dysmorphic disorder, hoarding disorder, trichotillomania, and excoriation disorder, panic attack, panic disorder, post-traumatic stress disorder, separation anxiety disorder, social phobia, specific phobia, substance-induced anxiety disorder and anxiety due to a general medical condition; substance-related disorders and addictive behaviors (including substance-induced delirium, persisting dementia, persisting amnestic disorder, psychotic disorder or anxiety disorder; tolerance, dependence or withdrawal from substances including alcohol, amphetamines, cannabis, cocaine, hallucinogens, inhalants, nicotine, opioids, phencyclidine, sedatives, hypnotics or anxiolytics); eating disorders such as obesity, bulimia nervosa, pica and compulsive eating disorders; persistent depressive disorder (PDD), major depressive disorder (MDD), as an adjunctive treatment MDD, major depressive disorder with anxious distress, MDD with mixed features (MDD-MF), MDD with melancholic features, MDD with atypical features, MDD with mood-congruent psychotic features, MDD with mood-incongruent psychotic features, MDD with catatonia, with peripartum onset, MDD with seasonal pattern, treatment resistant depression (TRD), and persistent depressive disorder (dysthymia), and are associated with depressed mood (sadness), poor concentration, insomnia, fatigue, appetite disturbances, excessive guilt and thoughts of suicide, premenstrual syndrome (PMS) and premenstrual dysphoric disorder (PDD), mood disorders due to a general medical condition, and substance-induced mood disorders; and sleep disorders including insomnia, disturbed sleep, jet lag, hypersomnia, cataplexy, sleep apnea, obstructive sleep apnea, REM sleep behavior disorder, Restless Leg Syndrome, periodic limb movement disorder, circadian rhythm sleep disorders, delayed sleep phase disorder, sleepwalking, night terrors, bed wetting, rapid eye movement sleep behavior disorder, shift work sleep disorder, excessive daytime sleepiness, non-24-hour sleep-wake disorder, sleep paralysis and narcolepsy.

Psychiatric disorders are pathological conditions of the brain characterized by identifiable symptoms that result in abnormalities in cognition, emotion or mood, or the highest integrative aspects of behavior. These disorders may vary in severity of symptoms, duration, and functional impairment. Psychiatric disorders afflict millions of people worldwide resulting in tremendous human suffering and economic burden due to lost productivity. Mood disorders are a type of psychiatric disorder often defined as a group of heterogeneous disorders characterized by pervasive mood disturbances, psychomotor dysfunction, and vegetative symptoms. Suicide, the most serious complication in patients with mood disorders, is the cause of death in 15 to 25% of untreated patients with mood disorders; unrecognized or inadequately treated depression contributes to 50 to 70% of all completed suicides.

The term “mood disorder” as used herein includes depression, major depression, major depressive disorder, mild depression, severe depression without psychosis, severe depression with psychosis, melancholia (formerly endogenous depression), atypical depression, dysthymic disorder, and manic depression.

In various embodiments, the neurological or psychiatric disease or disorder is a depressive disorder, such as, persistent depressive disorder (PDD), major depressive disorder (MDD), major depressive disorder with mixed features (MDD-MF), and treatment resistant depression (TRD).

Depression is an affective disorder, the pathogenesis of which cannot be explained by any single cause or theory. Unfortunately, treatment options for depressed patients who have suboptimal clinical responses to therapy with an antidepressant are limited. Approximately thirty percent (30%) of patients initiating antidepressant therapy show suboptimal or delayed clinical responses to the first-line antidepressant agents that are commonly used to treat depression.

Typically, if a patient exhibits suboptimal or delayed clinical response after several weeks of therapy with an antidepressant, the clinician's initial approach is to increase the dose of the antidepressant. If the patient's response remains unsatisfactory after increasing the dose, the most common approaches that many clinicians will pursue are: a) switching to another antidepressant; or b) adding a second antidepressant; or c) attempting an augmentation therapy by administering agents such as lithium carbonate, thyroid hormone (triiodothyronine), psychostimulants, modafinil, atypical antipsychotics, buspirone, or pindolol.

In its full syndromal expression_{;} clinical depression manifests as major depressive disorder, with episodic course and varying degrees of residual manifestations between episodes. The mood is typically depressed, irritable, and/or anxious. The patient may appear miserable, with furrowed brows, downturned corners of the mouth, slumped posture, poor eye contact, and monosyllabic (or absent) speech. The morbid mood may be accompanied by preoccupation with guilt, self-denigrating ideas, decreased ability to concentrate, indecisiveness, diminished interest in usual activities, social withdrawal, helplessness, hopelessness, and recurrent thoughts of death and suicide. Sleep disorders are common. In some, the morbid mood is so deep that tears dry up; the patient complains of an inability to experience usual emotions - including grief, joy, and pleasure—and of a feeling that the world has become colorless, lifeless, and dead.

Melancholia (formerly endogenous depression) is characterized by marked psychomotor slowing (of thinking and activity) or agitation (e.g., restlessness, wringing of the hands, pressure of speech), weight loss, irrational guilt, and loss of the capacity to experience pleasure. Mood and activity vary diurnally, with a nadir in the morning. Most melancholic patients complain of difficulty falling asleep, multiple arousals, and insomnia in the middle of the night or early morning. Sexual desire is often diminished or lost. Amenorrhea can occur. Anorexia and weight loss may lead to emaciation and secondary disturbances in electrolyte balance.

In atypical depression, reverse vegetative features dominate the clinical presentation; they include anxious-phobic symptoms, evening worsening, initial insomnia, hypersomnia that often extends into the day, and hyperphagia with weight gain.

In dysthymic disorder (aka persistent depressive disorder), depressive symptoms typically begin insidiously in childhood or adolescence and pursue an intermittent or low-grade course over many years or decades; major depressive episodes may complicate it (double depression). In pure dysthymia, depressive manifestations occur at a subthreshold level and overlap considerably with those of a depressive temperament: habitually gloomy, pessimistic, humorless, or incapable of fun; passive and lethargic; introverted; skeptical, hypercritical, or complaining; self-critical, self-reproaching, and self-derogatory; and preoccupied with inadequacy, failure, and negative events.

It is to be understood that TRD is a term used in clinical psychiatry to describe cases of major depressive disorder (MDD) that do not respond adequately to appropriate courses of adequate dose and duration of at least two antidepressants.

In various embodiments, a depressive disorder is associated with acute suicidality or suicide ideation. The United States Food and Drug Administration has adopted a “black box” label warning indicating that antidepressants may increase the risk of suicidal thinking and behavior in some children, adolescents and young adults (up to age 24) with a depressive disorder such as MDD. In various embodiments, it is believed that the compositions and methods of the present inventions do not increase the risk of suicidal thinking and/or behavior in children, adolescents and/or young adults with a depressive disorder, e.g., with MDD. In various embodiments, the present inventions provide medicaments for and provide methods of treating one or more symptoms of a depressive disorder (e.g., MDD) in children, adolescents and/or young adults without increasing the risk of suicidal thinking and/or behavior.

In various embodiments, the neurological or psychiatric disease or disorder is schizophrenia. Schizophrenia is a disorder of unknown origin, which usually appears for the first time in early adulthood and is marked by characteristics such as psychotic symptoms, phasic progression and development, and/or deterioration in social behavior and professional capability. Characteristic psychotic symptoms are disorders of thought content (e.g., multiple, fragmentary, incoherent, implausible or simply delusional contents, or ideas of persecution) and of mentality (e.g., loss of association, flight of imagination, incoherence up to incomprehensibility), as well as disorders of perceptibility (e.g., hallucinations), emotions (e.g., superficial or inadequate emotions), self-perceptions, intentions, impulses, and/or inter-human relationships, and psychomotoric disorders (e.g., catatonia). Other symptoms are also associated with this disorder. Schizophrenia is classified into subgroups: the paranoid type, characterized by delusions and hallucinations and absence of thought disorder, disorganized behavior, and affective flattening; the disorganized type, also named “hebephrenic schizophrenia,” in which thought disorder and flat affect are present together; the catatonic type, in which prominent psychomotor disturbances are evident, and symptoms may include catatonic stupor and waxy flexibility; and the undifferentiated type, in which psychotic symptoms are present but the criteria for paranoid, disorganized, or catatonic types have not been met. The symptoms of schizophrenia normally manifest themselves in three broad categories: positive, negative and cognitive symptoms. Positive symptoms are those which represent an “excess” of normal experiences, such as hallucinations and delusions. Negative symptoms are those where the patient suffers from a lack of normal experiences, such as anhedonia and lack of social interaction. The cognitive symptoms relate to cognitive impairment in schizophrenics, such as lack of sustained attention and deficits in decision making.

Accordingly, in various embodiments, the neurological or psychiatric disease or disorder is one or more of schizotypal (personality) disorder, delusional disorder, brief psychotic disorder, schizophreniform disorder, schizophrenia, substance/medication-induced psychotic disorder, psychotic disorder due to another medical condition, other specified schizophrenia spectrum and other psychotic disorder, unspecified schizophrenia spectrum, and other psychotic disorder.

In various embodiments, the neurological or psychiatric disease or disorder is anxiety disorder. Anxiety disorders are characterized by fear, worry, and uneasiness, usually generalized and unfocused as an overreaction to a situation. Anxiety disorders differ in the situations or types of objects that induce fear, anxiety, or avoidance behavior, and the associated cognitive ideation. Anxiety differs from fear in that anxiety is an emotional response to a perceived future threat while fear is associated with a perceived or real immediate threat. They also differ in the content of the associated thoughts or beliefs. Examples of anxiety disorders include separation anxiety disorder, selective mutism, specific phobia, social anxiety disorder (social phobia), panic disorder, panic attack specifier, agoraphobia, generalized anxiety disorder, substance/medication-induced anxiety disorder, anxiety disorder due to another medical condition, illness anxiety disorder, social (pragmatic) communication disorder, other specified anxiety disorder, and unspecified anxiety disorder; stressor-related disorders, including reactive attachment disorder, disinhibited social engagement disorder, posttraumatic stress disorder (PTSD), acute stress disorder, and adjustment disorders.

In various embodiments, the neurological or psychiatric disease or disorder is a sleep disorder including those sleep disorders which are produced by psychiatric conditions, including, but not limited to, insomnia, disturbed sleep, jet lag, hypersomnia, cataplexy, sleep related disorder (e.g., sleep apnea, insomnia, narcolepsy, cataplexy), obstructive sleep apnea, REM sleep behavior disorder, Restless Leg Syndrome, periodic limb movement disorder, circadian rhythm sleep disorders, delayed sleep phase disorder, sleepwalking, night terrors, bed wetting, rapid eye movement sleep behavior disorder, shift work sleep disorder, excessive daytime sleepiness, non-24-hour sleep-wake disorder, sleep paralysis and narcolepsy.

In various embodiments, the neurological and/or psychiatric disease or disorders are obsessive-compulsive disorder and related disorders (e.g., body dysmorphic disorder, hoarding disorder, trichotillomania, excoriation disorder).

In various embodiments, the neurological and/or psychiatric diseases or disorders are disruptive, impulse-control, and conduct disorders including oppositional defiant disorder, intermittent explosive disorder, conduct disorder, antisocial personality disorder, pyromania, kleptomania, other specified disruptive, impulse-control, and conduct disorder, unspecified disruptive, impulse-control, and conduct disorder.

In various embodiments, the compositions, formulations, methods and medicaments may be used in combination with other therapies. Suitable therapies include, but are not limited to, psychotherapy, cognitive behavioral therapy, electroconvulsive therapy, transcranial magnetic stimulation, vagus nerve stimulation, and deep-brain stimulation.

Aspects, embodiments, and features may be further understood from the following examples, which should not be construed as limiting the scope of the inventions.

### EXAMPLE 1: In Vitro Assays of Dopamine D₂ and Serotonin 5-HT7 Affinities

Amisulpride enantiomers and racemic amisulpride were tested for affinity to Dopamine Des receptors recombinantly expressed in human Chinese Hamster Ovary (CHO) cells by radioligand binding techniques (Eurofins Panlabs, Inc.). The receptors' Bₘₐₓ value was 1.6 pmole/mg protein. The radioligand was [3H]Spiperone at 0.16 nM concentration with 0.090 nM dissociation constant (Kd, historical value under identical laboratory conditions). The incubation buffer was 50 mM Tris-HCl, pH 7.4, 1.4 mM ascorbic acid, 0.001% BSA, and 150 mM NaCl. The amisulpride compound under study (e.g., enantiomeric amisulprides and racemic amisulpride) was dissolved in dimethyl sulfoxide (DMSO) and added to the assay wells for a 1% final concentration. Percent inhibition values of specific binding by amisulpride enantiomers and racemic amisulpride were generated with 12 serial dilutions from 10 micromolar down to 3 nM final concentrations. Each concentration was tested in duplicate. Amisulpride enantiomer affinities and racemic amisulpride affinities for dopamine D₂ receptors are based on the average of 3 independent experiments. Affinities were calculated with the Cheng-Prusoff equation and the observed IC50 of the tested compound, the concentration of radioligand employed in the assay, and the historical value for the Kd of the ligand (obtained experimentally).

Amisulpride enantiomers and racemic amisulpride were tested for affinity to Serotonin 5-HT₇ receptors recombinantly expressed in human CHO-Kl cells by radioligand binding techniques (Eurofins Panlabs, Inc.). The receptors' Bmax value was 0.95 pmole/mg protein. The radioligand is [3H]Lysergic acid diethylamide (LSD) at 5.5 nM concentration with 7.40 nM dissociation constant (Kd, historical value under identical laboratory conditions). The incubation buffer was 50 mM Tris-HC1, pH 7.4, 10 mM MgCl₂, 0.5 mM EDTA. The amisulpride compound under study (e.g., enantiomeric amisulprides and racemic amisulpride) was dissolved in DMSO and added to the assay wells for a 1% final concentration. Percent inhibition values of specific binding by amisulpride enantiomers and racemic amisulpride were generated with 12 serial dilutions from 10 micromolar down to 3 nM final concentrations. Each concentration was tested in duplicate. Amisulpride enantiomer affinities and racemic amisulpride affinities for serotonin 5-HT7 receptors are based on the average of 3 independent experiments. Affinities were calculated with the Cheng-Prusoff equation and the observed IC50 of the tested compound, the concentration of radioligand employed in the assay, and the historical value for the Kd of the ligand (obtained experimentally).

Percent inhibition of specific binding was determined as a function of test drug concentration (i.e., (R)-amisulpride (S)-amisulpride, and racemic amisulpride). It was discovered that there are distinct pharmacological activities with the potential for combined clinical benefit which reside in opposite enantiomers.

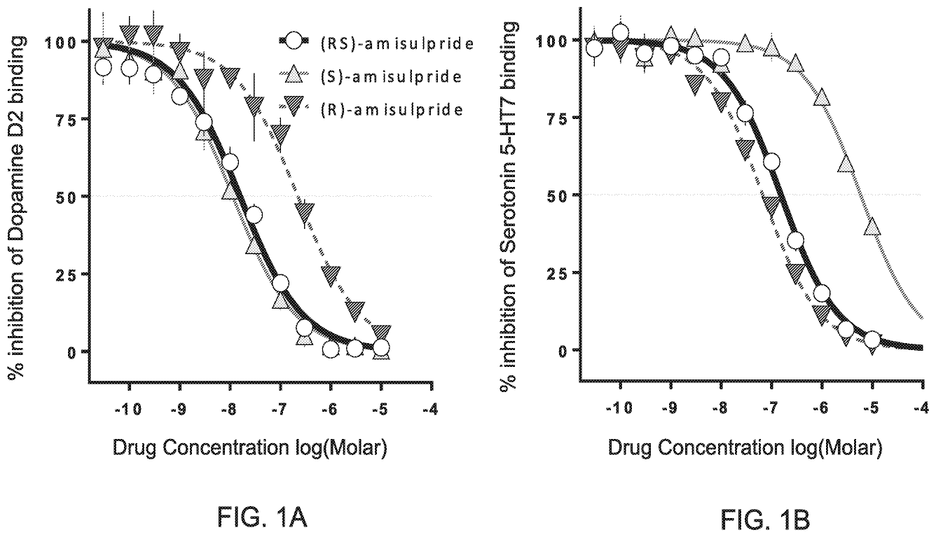

Referring to FIG. 1A, depicted is the data on the % inhibition of dopamine D₂ binding of Example 1 for (R)-amisulpride (downward triangle), (S)-amisulpride (upward triangle), and racemic amisulpride (circle). The vertical bars represent±1 standard deviation from the 3 independent determinations. FIG. 1A illustrates that the (S)-enantiomer is the more potent enantiomer for dopamine D₂ receptors.

Referring to FIG. 1B, depicted is the data on the % inhibition of serotonin 5-HT7 binding of Example 1 for (R)-amisulpride (downward triangle), (S)-amisulpride (upward triangle), and racemic amisulpride (circle). The vertical bars represent ±1 standard deviation from the 3 independent determinations. FIG. 1B illustrates that the (R)-enantiomer is more potent in inhibiting binding to serotonin 5-HT₇ receptors.

Table 10 summarizes inhibitor constant (Ki) values in nM determined in vitro by radioligand binding of racemic. Human dopamine D₂ receptors or human serotonin 5-HT₇ receptors were expressed in CHO cells or CHO-K1 cells, respectively. Standard error of the mean is presented based on multiple, independent determinations.

TABLE 10

| | Racemic (50:50) |
|---|---|
| Dopamine D2 | 7.1 ± 0.26 |
| Serotonin 5-HT7 | 89 ± 2 |
| 5-HT7/D2 | 13 |

Example 1 shows that the (R)-enantiomer is highly stereoselective for serotonin 5-HT7 receptors such that the 5-HT7 antagonism of amisulpride resides almost exclusively in the (R)-enantiomer and that the (S)-enantiomer is highly stereoselective for dopamine D₂ receptors such that the D₂ antagonism of racemic amisulpride resides predominantly in the (S)-enantiomer. Referring to again to FIG. 1A, the D₂ antagonism of (S)-amisulpride was determined to be about 20 fold that of the (R)-amisulpride, and referring to again to FIG. 1B, the 5-HT7 antagonism of (R)-amisulpride was determined to be about 300 fold that of the (S)-amisulpride.

The present inventions also include the following aspects and embodiments. The following aspects and embodiments are listed with numerical references for convenience in exposition and reference, such numerical listing and reference is not meant to be construed in a limiting sense.

Embodiment 1, a pharmaceutical composition in a solid oral dosage form, the solid oral dosage form comprising, racemic amisulpride, or pharmaceutically acceptable salts thereof; and

an extended release agent in an amount between about 10% to about 50% by total solid oral dosage form weight, wherein when administered to a subject population, said pharmaceutical composition provides a population average maximum QT interval prolongation relative to baseline over the time period of 12 hours after administration of is less than about 0.45 milliseconds (ms) per 10 mg of amisulpride.

Embodiment 2, the pharmaceutical composition of embodiment 1, wherein the population average maximum QT interval prolongation relative to baseline is the population average maximum QTcF interval prolongation relative to baseline.

Embodiment 3, the pharmaceutical composition of embodiment 1, wherein the population average maximum QT interval prolongation relative to baseline is less than about 0.40 milliseconds (ms) per 10 mg of amisulpride.

Embodiment 4, the pharmaceutical composition of embodiment 1, wherein the population average maximum QT interval prolongation relative to baseline is less than about 0.35 milliseconds (ms) per 10 mg of amisulpride.

Embodiment 5, the pharmaceutical composition of embodiment 1, wherein the population average maximum QT interval prolongation relative to baseline is less than about 0.30 milliseconds (ms) per 10 mg of amisulpride.

Embodiment 6, the pharmaceutical composition of embodiment 1, wherein the population average maximum QT interval prolongation relative to baseline is: (a) less than about 0.25 milliseconds (ms) per 10 mg of amisulpride; or (b) less than about 0.20 milliseconds (ms) per 10 mg of amisulpride; or (c) less than about 0.15 milliseconds (ms) per 10 mg of amisulpride; or (d) less than about 0.10 milliseconds (ms) per 10 mg of amisulpride; or (e) less than about 0.05 milliseconds (ms) per 10 mg of amisulpride or (f) less than about 0.02 milliseconds (ms) per 10 mg of amisulpride.

Embodiment 7, the pharmaceutical composition of embodiment 1, wherein said solid oral dosage form is a tablet.

Embodiment 8, the pharmaceutical composition of embodiment 7, wherein the amount of racemic amisulpride, or pharmaceutically acceptable salts thereof, is: (a) about 100 mg; or (b) about 150 mg; or (c) about 200 mg; or (d) about 250 mg; or (e) about 300 mg by weight of free base.

Embodiment 9, the pharmaceutical composition of embodiment 1, wherein the extended release agent comprises a matrix forming agent.

Embodiment 10, the pharmaceutical composition of embodiment 9, wherein the matrix forming agent comprises one or more cellulosic ethers.

Embodiment 11, the pharmaceutical composition of embodiment 1, wherein the extended release agent is in an amount: (a) between about 30% and about 50% by total solid oral dosage form weight; or (b) between about 20% and about 40% by total solid oral dosage form weight; or (c) between about 20% and about 35% by total solid oral dosage form weight.

Embodiment 12, the pharmaceutical composition of embodiment 1, wherein the extended release agent comprises hydroxypropyl methylcellulose in an amount between about 20% to about 35% by total solid oral dosage form weight.

Embodiment 13, the pharmaceutical composition of embodiment 1, wherein the solid oral dosage form comprises by total solid oral dosage form weight: between about 35% to about 55% of said racemic amisulpride, between about 10% to about 50% of a pharmaceutically acceptable filler, and between about 20% to about 35% of the extended release agent.

Embodiment 14, a method of treating a psychiatric disorder selected from schizophrenia, negative symptoms of schizophrenia, persistent depressive disorder (PDD), treatment resistant depression (TRD), major depressive disorder (MDD) and major depressive disorder with mixed features (MDD-MF), and depression comprising administering the pharmaceutical composition of embodiment 1.

Embodiment 15, the method of embodiment 14, wherein the psychiatric disorder is one or more of schizophrenia and negative symptoms of schizophrenia.

Embodiment 16, the method of embodiment 14, wherein the psychiatric disorder is one or more of major depressive disorder (MDD) and major depressive disorder with mixed features (MDD-MF).

Embodiment 17, the method of embodiment 14, comprising administering the solid oral dosage once per day in a total daily amount of between about 100 mg to about 1200 mg of said racemic amisulpride by weight of free base.

Embodiment 18, a pharmaceutical composition in a solid oral dosage form, the solid oral dosage form comprising, 200 mg of racemic amisulpride; and an extended release agent in an amount between about 10% to about 50% by total solid oral dosage form weight, wherein when administered to a subject population provides a population average maximum QTcF interval prolongation relative to baseline over the time period of 12 hours after administration of: (a) less than about 10 milliseconds (ms); or (b) less than about 9 milliseconds (ms); or (c) less than about 8 milliseconds (ms); or (d) less than about 7 milliseconds (ms); or (e) less than about 6 milliseconds (ms); or (f) less than about 5 milliseconds (ms).

Embodiment 19, the pharmaceutical composition of embodiment 18, wherein the population average maximum QT interval prolongation relative to baseline is the population average maximum QTcF interval prolongation relative to baseline.

Embodiment 20, the pharmaceutical composition of embodiment 18, wherein said solid oral dosage form is a tablet.

Embodiment 21, the pharmaceutical composition of embodiment 20, wherein the solid oral dosage when administered provides a blood plasma population geometric mean Cmax of (a) less than about 350 ng/mL; (b) less than about 300 ng/mL; or (c) less than about 250 ng/mL.

Embodiment 22, the pharmaceutical composition of embodiment 18, wherein the extended release agent comprises a matrix forming agent.

Embodiment 23, the pharmaceutical composition of embodiment 22, wherein the matrix forming agent comprises one or more cellulosic ethers.

Embodiment 24, the pharmaceutical composition of embodiment 18, wherein the extended release agent is in an amount: (a) between about 30% and about 50% by total solid oral dosage form weight; or (b) between about 20% and about 40% by total solid oral dosage form weight; or (c) between about 20% and about 35% by total solid oral dosage form weight.

Embodiment 25, the pharmaceutical composition of embodiment 18, wherein the extended release agent comprises hydroxypropyl methylcellulose in an amount between about 20% to about 35% by total solid oral dosage form weight.

Embodiment 26, a method of treating a psychiatric disorder selected from schizophrenia, negative symptoms of schizophrenia, persistent depressive disorder (PDD), treatment resistant depression (TRD), major depressive disorder (MDD) and major depressive disorder with mixed features (MDD-MF), and depression comprising administering the pharmaceutical composition of embodiment 18.

Embodiment 27, the method of embodiment 26, wherein the psychiatric disorder is one or more of schizophrenia and negative symptoms of schizophrenia.

Embodiment 28, the method of embodiment 26, wherein the psychiatric disorder is one or more of major depressive disorder (MDD) and major depressive disorder with mixed features (MDD-MF).

Embodiment 29, the method of embodiment 26, comprising administering the solid oral dosage form once per day in a total daily amount of between about 100 mg to about 1200 mg of said racemic amisulpride by weight of free base

Embodiment 30, a method of treating a psychiatric disorder comprising: administering racemic amisulpride as a solid oral dosage form to a subject, the solid oral dosage form comprising

racemic amisulpride, or pharmaceutically acceptable salts thereof, and an extended release agent in an amount between about 10% to about 50% by total solid oral dosage form weight;

wherein said administration provides a subject population average maximum QT interval prolongation relative to baseline that is less than about 0.4 milliseconds (ms) per 10 mg of amisulpride.

Embodiment 31, the method of embodiment 30, wherein the population average maximum QT interval prolongation relative to baseline is the population average maximum QTcF interval prolongation relative to baseline over the time period of 12 hours after said administration.

Embodiment 32, the method of embodiment 30, wherein the population average maximum QT interval prolongation relative to baseline is less than about 0.35 milliseconds (ms) per 10 mg of amisulpride.

Embodiment 33, the method of embodiment 30, wherein the population average maximum QT interval prolongation relative to baseline is less than about 0.3 milliseconds (ms) per 10 mg of amisulpride.

Embodiment 34, the method of embodiment 30, wherein the population average maximum QT interval prolongation relative to baseline is less than about 0.25 milliseconds (ms) per 10 mg of amisulpride.

Embodiment 35, the method of embodiment 30, wherein said administration is once per day.

Embodiment 36, the method of embodiment 30, wherein said solid oral dosage form is a tablet.

Embodiment 37, the method of embodiment 30, wherein said administration is between about 100 mg per day of amisulpride by weight of free base to about 1200 mg per day of amisulpride by weight of free base.

Embodiment 38, the method of embodiment 37, wherein said psychiatric disorder is selected from schizophrenia, negative symptoms of schizophrenia, persistent depressive disorder (PDD), treatment resistant depression (TRD), major depressive disorder (MDD) and major depressive disorder with mixed features (MDD-MF), and depression.

Embodiment 39, the method of embodiment 38, wherein the psychiatric disorder is one or more of schizophrenia and negative symptoms of schizophrenia.

Embodiment 40, the method of embodiment 38, wherein the psychiatric disorder is one or more of major depressive disorder (MDD) and major depressive disorder with mixed features (MDD-MF).

Embodiment 41, the method of embodiment 30, wherein the extended release agent comprises a matrix forming agent.

Embodiment 42, the method of embodiment 41, wherein the matrix forming agent comprises one or more cellulosic ethers.

Embodiment 43, the method of embodiment 30, wherein the extended release agent is in an amount: (a) between about 30% and about 50% by total solid oral dosage form weight; or (b) between about 20% and about 40% by total solid oral dosage form weight; or (c) between about 20% and about 35% by total solid oral dosage form weight.

Embodiment 44, the method of embodiment 30, wherein the extended release agent comprises hydroxypropyl methylcellulose in an amount between about 20% to about 35% by total solid oral dosage form weight.

Embodiment 45, the method of embodiment 30, wherein the solid oral dosage form comprises by total solid oral dosage form weight: between about 35% to about 55% of said racemic amisulpride, between about 10% to about 50% of a pharmaceutically acceptable filler, and between about 20% to about 35% of the extended release agent.

Embodiment 46, a pharmaceutical composition in a solid oral dosage form, the solid oral dosage form comprising, racemic amisulpride, or pharmaceutically acceptable salts thereof; and

an extended release agent in an amount between about 10% to about 50% by total solid oral dosage form weight, wherein the solid oral dosage form when dissolution tested using a two-stage in vitro gastrointestinal simulation dissolution test releases less than about 30% of the amisulpride after 1 hour, releases more than about 30% and less than about 55% of the amisulpride after 3 hours, and releases more than about 50% and less than about 90% of the amisulpride after 6 hours.

Embodiment 47, the pharmaceutical composition of embodiment 46, wherein the solid oral dosage form when dissolution tested using a two-stage in vitro gastrointestinal simulation dissolution test releases less than about 20% of the amisulpride after 1 hour.

Embodiment 48, the pharmaceutical composition of embodiment 46, wherein the solid oral dosage form when dissolution tested using a two-stage in vitro gastrointestinal simulation dissolution test releases more than about 30% and less than about 50% of the amisulpride after 3 hours.

Embodiment 49, the pharmaceutical composition of embodiment 46, wherein the solid oral dosage form when dissolution tested using a two-stage in vitro gastrointestinal simulation dissolution test releases more than about 30% and less than about 40% of the amisulpride after 3 hours.

Embodiment 50, the pharmaceutical composition of embodiment 46, wherein the solid oral dosage form when dissolution tested using a two-stage in vitro gastrointestinal simulation dissolution test releases more than about 50% and less than about 80% of the amisulpride after 6 hours.

Embodiment 51, the pharmaceutical composition of embodiment 46, wherein the solid oral dosage form when dissolution tested using a two-stage in vitro gastrointestinal simulation dissolution test releases more than about 60% and less than about 90% of the amisulpride after 6 hours.

Embodiment 52, the pharmaceutical composition of embodiment 46, wherein the two-stage gastrointestinal simulation dissolution test is conducted in a paddle apparatus substantially in accord with that described in Table 4 in the paddle apparatus described in United States Pharmacopeia Convention (USP) Apparatus 2 of Chapter 711 Dissolution; USP41-NF36 General Chapter <711>.

Embodiment 53, the pharmaceutical composition of embodiment 46, wherein said solid oral dosage form is a tablet.

Embodiment 54, the pharmaceutical composition of embodiment 53, wherein the amount of racemic amisulpride, or pharmaceutically acceptable salts thereof, is: (a) about 100 mg; or (b) about 150 mg; or (c) about 200 mg; or (d) about 250 mg; or (e) about 300 mg by weight of free base.

Embodiment 55, the pharmaceutical composition of embodiment 54, wherein the extended release agent comprises a matrix forming agent.

Embodiment 56, the pharmaceutical composition of embodiment 55, wherein the matrix forming agent comprises one or more cellulosic ethers.

Embodiment 57, the pharmaceutical composition of embodiment 46, wherein the extended release agent is in an amount: (a) between about 30% and about 50% by total solid oral dosage form weight; or (b) between about 20% and about 40% by total solid oral dosage form weight; or (c) between about 20% and about 35% by total solid oral dosage form weight.

Embodiment 58, the pharmaceutical composition of embodiment 46, wherein the extended release agent comprises hydroxypropyl methylcellulose in an amount between about 20% to about 35% by total solid oral dosage form weight.

Embodiment 59, a method of treating a psychiatric disorder selected from schizophrenia, negative symptoms of schizophrenia, persistent depressive disorder (PDD), treatment resistant depression (TRD), major depressive disorder (MDD) and major depressive disorder with mixed features (MDD-MF), and depression comprising administering the pharmaceutical composition of embodiment 46.

Embodiment 60, the method of embodiment 59, wherein the psychiatric disorder is one or more of schizophrenia and negative symptoms of schizophrenia.

Embodiment 61, the method of embodiment 60, wherein the psychiatric disorder is one or more of major depressive disorder (MDD) and major depressive disorder with mixed features (MDD-MF).

Embodiment 62, the method of embodiment 59, comprising administering the solid oral dosage form once per day in a total daily amount of between about 100 mg to about 1200 mg of said racemic amisulpride by weight of free base.

Embodiment 63, a pharmaceutical composition in a solid oral dosage form, the solid oral dosage form comprising, racemic amisulpride, or pharmaceutically acceptable salts thereof; and

an extended release agent in an amount between about 10% to about 50% by total solid

oral dosage form weight, wherein the solid oral dosage form when dissolution tested using the two-stage in vitro dissolution test described in Table 4 in the paddle apparatus described in United States Pharmacopeia Convention (USP) Apparatus 2 of Chapter 711 Dissolution; USP41-NF36 General Chapter <711> Dissolution has a dissolution profile substantially the same as: (a) the profile of Tab 1D in FIG. 2; or (b) the profile of Tab 2D in FIG. 2.

Embodiment 64, the pharmaceutical composition of embodiment 63, wherein said solid oral dosage form is a tablet.

Embodiment 65, the pharmaceutical composition of embodiment 63, wherein the amount of racemic amisulpride, or pharmaceutically acceptable salts thereof, is: (a) about 100 mg; or (b) about 150 mg; or (c) about 200 mg; or (d) about 250 mg; or (e) about 300 mg by weight of free base.

Embodiment 66, the pharmaceutical composition of embodiment 63, wherein the extended release agent comprises a matrix forming agent.

Embodiment 67, the pharmaceutical composition of embodiment 66, wherein the matrix forming agent comprises one or more cellulosic ethers.

Embodiment 68, the pharmaceutical composition of embodiment 63, wherein the extended release agent is in an amount: (a) between about 30% and about 50% by total solid oral dosage form weight; or (b) between about 20% and about 40% by total solid oral dosage form weight; or (c) between about 20% and about 35% by total solid oral dosage form weight.

Embodiment 69, the pharmaceutical composition of embodiment 63, wherein the extended release agent comprises hydroxypropyl methylcellulose in an amount between about 20% to about 35% by total solid oral dosage form weight.

Embodiment 70, a method of treating a psychiatric disorder selected from schizophrenia, negative symptoms of schizophrenia, persistent depressive disorder (PDD), treatment resistant depression (TRD), major depressive disorder (MDD) and major depressive disorder with mixed features (MDD-MF), and depression comprising administering the pharmaceutical composition of embodiment 63.

Embodiment 71, the method of embodiment 70, wherein the psychiatric disorder is one or more of schizophrenia and negative symptoms of schizophrenia.

Embodiment 72, the method of embodiment 70, wherein the psychiatric disorder is one or more of major depressive disorder (MDD) and major depressive disorder with mixed features (MDD-MF).

Embodiment 73, the method of embodiment 70, comprising administering the solid oral dosage form once per day in a total daily amount of between about 100 mg to about 1200 mg of said racemic amisulpride by weight of free base.

Embodiment 74, the solid oral dosage form of any one of embodiments 1, 18, 30, 46, and 63, wherein the solid oral dosage form comprises one or more of (a) a filler; (b) a binder; and (c) a lubricant.

Embodiment 75, the solid oral dosage form of embodiment 75, wherein the lubricant comprises magnesium stearate.

Embodiment 76, the solid oral dosage form of embodiment 75, wherein the filler comprises D-mannitol and wherein the solid oral dosage form comprises between about 0.5% to about 2% by total tablet weight of a binder comprising polyvinyl alcohol.

Embodiment 77, the solid oral dosage form of any one of embodiments 7, 20, 36, 53, and 64, wherein the tablet comprises: a granular component admixed with an extra-granular component, the granular component comprising the racemic amisulpride and a binder; and

the extra-granular component comprising, an extended release agent.

Embodiment 78, the pharmaceutical composition of embodiment 77, wherein the granular component comprises one or more of (a) a filler; and (b) a binder.

Embodiment 79, the pharmaceutical composition of embodiment 78, wherein the granules comprise: (a) between about 60% to about 80% by weight of amisulpride, between about 10% to about 30% by weight of filler, and between about 1% to about 5% by weight of binder; or (b) between about 70% to about 80% by weight of amisulpride, between about 20% to about 25% by weight of filler, and between about 1% to about 5% by weight of binder.

Embodiment 80, the pharmaceutical composition of embodiment 78, wherein the granular component comprises: between about 73% to about 78% by weight of amisulpride, between about 10% to about 12% by weight of a D-mannitol, between about 10% to about 12% by weight of a pregelatinized starch, and between about 1% to about 3% by weight of polyvinyl alcohol; based on the weight of the granular component.

Embodiment 81, the pharmaceutical composition of embodiment 77, wherein the extragranular component comprises one or more of (a) a filler; (b) a binder; and (c) a lubricant.

Embodiment 82, the pharmaceutical composition of embodiment 77, wherein the tablet (granules plus extragranular component) comprises: (a) between about 20% to about 70% by total tablet weight of granules of extended release agent; or (b) between about 10% to about 50% by total tablet weight of extended release agent.

Embodiment 83, the pharmaceutical composition of embodiment 77, wherein the tablet (granules plus extragranular component) comprises: (a) a combined amount of filler in both granular and extragranular between about 6% to about 60% by total tablet weight; or (b) a combined amount of filler in both granular and extragranular between about 10% to about 50% by total tablet weight.

Embodiment 84, the pharmaceutical composition of embodiment 77, wherein the tablet (granules plus extragranular component) comprises between about 1% to about 2% by total tablet weight of a lubricant.

Embodiment 85, the pharmaceutical composition of embodiment 84, wherein the lubricant is magnesium stearate.

Embodiment 86, the pharmaceutical composition of embodiment 77, wherein the tablet (granules plus extragranular component) comprises: (a) between about 30% to about 50% by total tablet weight of a D-mannitol, and about 20% to about 35% by total tablet weight of hydroxypropyl methylcellulose; or (b) between about 20% to about 30% by total tablet weight of a D-mannitol, and about 20% to about 35% by total tablet weight of hydroxypropyl methylcellulose; or (c) between about 10% to about 20% by total tablet weight of a D-mannitol, and about 20% to about 35% by total tablet weight of hydroxypropyl methylcellulose.

Although the invention has been described with reference to a specific embodiment this description is not meant to be construed in a limiting sense. The invention being thus described, it is apparent that the same can be varied in many ways. Such variations are not to be regarded as a departure from the spirit and scope of the present invention, and all such modifications, alternatives, and equivalents as would be obvious to one skilled in the art are intended to be included within the scope of the following claims.

What is claimed is:

1. A pharmaceutical composition in a solid oral dosage form, the solid oral dosage form comprising,
racemic amisulpride, or pharmaceutically acceptable salts thereof; and
an extended release agent in an amount between about 10% to about 50% by total solid oral dosage form weight,
wherein when administered to a subject population, said pharmaceutical composition provides a population average maximum QT interval prolongation relative to baseline over the time period of 12 hours after administration of is less than about 0.45 milliseconds (ms) per 10 mg of amisulpride.

2. The pharmaceutical composition of claim 1, wherein the population average maximum QT interval prolongation relative to baseline is the population average maximum QTcF interval prolongation relative to baseline.

3. The pharmaceutical composition of claim 1, wherein the population average maximum QT interval prolongation relative to baseline is less than about 0.40 milliseconds (ms) per 10 mg of amisulpride.

4. The pharmaceutical composition of claim 1, wherein the population average maximum QT interval prolongation relative to baseline is less than about 0.35 milliseconds (ms) per 10 mg of amisulpride.

5. The pharmaceutical composition of claim 1, wherein the population average maximum QT interval prolongation relative to baseline is less than about 0.30 milliseconds (ms) per 10 mg of amisulpride.

6. The pharmaceutical composition of claim 1, wherein the population average maximum QT interval prolongation relative to baseline is: (a) less than about 0.25 milliseconds (ms) per 10 mg of amisulpride; or (b) less than about 0.20 milliseconds (ms) per 10 mg of amisulpride; or (c) less than about 0.15 milliseconds (ms) per 10 mg of amisulpride; or (d) less than about 0.10 milliseconds (ms) per 10 mg of amisulpride;
or (e) less than about 0.05 milliseconds (ms) per 10 mg of amisulpride or (0 less than about 0.02 milliseconds (ms) per 10 mg of amisulpride.

7. The pharmaceutical composition of claim 1, wherein said solid oral dosage form is a tablet.

8. The pharmaceutical composition of claim 7, wherein the amount of racemic amisulpride, or pharmaceutically acceptable salts thereof, is: (a) about 100 mg; or (b) about 150 mg; or (c) about 200 mg; or (d) about 250 mg; or (e) about 300 mg by weight of free base.

9. The pharmaceutical composition of claim 1, wherein the extended release agent comprises a matrix forming agent.

10. The pharmaceutical composition of claim 9, wherein the matrix forming agent comprises one or more cellulosic ethers.

11. The pharmaceutical composition of claim 1, wherein the extended release agent is in an amount: (a) between about 30% and about 50% by total solid oral dosage form weight; or (b) between about 20% and about 40% by total solid oral dosage form weight; or (c) between about 20% and about 35% by total solid oral dosage form weight.

12. The pharmaceutical composition of claim 1, wherein the extended release agent comprises hydroxypropyl methylcellulose in an amount between about 20% to about 35% by total solid oral dosage form weight.

13. The pharmaceutical composition of claim 1, wherein the solid oral dosage form comprises by total solid oral dosage form weight:
between about 35% to about 55% of said racemic amisulpride,
between about 10% to about 50% of a pharmaceutically acceptable filler, and
between about 20% to about 35% of the extended release agent.

14. A method of treating a psychiatric disorder selected from schizophrenia, negative symptoms of schizophrenia, persistent depressive disorder (PDD), treatment resistant depression (TRD), major depressive disorder (MDD) and major depressive disorder with mixed features (MDD-MF), and depression comprising administering the pharmaceutical composition of claim 1.

15. The method of claim 14, wherein the psychiatric disorder is one or more of schizophrenia and negative symptoms of schizophrenia.

16. The method of claim 14, wherein the psychiatric disorder is one or more of major depressive disorder (MDD) and major depressive disorder with mixed features (MDD-MF).

17. The method of claim 14, comprising administering the solid oral dosage once per day in a total daily amount of between about 100 mg to about 1200 mg of said racemic amisulpride by weight of free base.

18. A pharmaceutical composition in a solid oral dosage form, the solid oral dosage form comprising,
200 mg of racemic amisulpride; and
an extended release agent in an amount between about 10% to about 50% by total solid oral dosage form weight,
wherein when administered to a subject population provides a population average maximum QTcF interval prolongation relative to baseline over the time period of 12 hours after administration of: (a) less than about 10 milliseconds (ms); or (b) less than about 9 milliseconds (ms); or (c) less than about 8 milliseconds (ms); or (d) less than about 7 milliseconds (ms); or (e) less than about 6 milliseconds (ms); or (f) less than about 5 milliseconds (ms).

19. The pharmaceutical composition of claim 18, wherein the population average maximum QT interval prolongation relative to baseline is the population average maximum QTcF interval prolongation relative to baseline.

20. The pharmaceutical composition of claim 18, wherein said solid oral dosage form is a tablet.

21. The pharmaceutical composition of claim 20, wherein the solid oral dosage when administered provides a blood plasma population geometric mean Cmax of (a) less than about 350 ng/mL; (b) less than about 300 ng/mL; or (c) less than about 250 ng/mL.

22. The pharmaceutical composition of claim 18, wherein the extended release agent comprises a matrix forming agent.

23. The pharmaceutical composition of claim 22, wherein the matrix forming agent comprises one or more cellulosic ethers.

24. The pharmaceutical composition of claim 18, wherein the extended release agent is in an amount: (a) between about 30% and about 50% by total solid oral dosage form weight; or (b) between about 20% and about 40% by total solid oral dosage form weight; or (c) between about 20% and about 35% by total solid oral dosage form weight.

25. The pharmaceutical composition of claim 18, wherein the extended release agent comprises hydroxypropyl methylcellulose in an amount between about 20% to about 35% by total solid oral dosage form weight.

26. A method of treating a psychiatric disorder selected from schizophrenia, negative symptoms of schizophrenia, persistent depressive disorder (PDD), treatment resistant depression (TRD), major depressive disorder (MDD) and major depressive disorder with mixed features (MDD-MF), and depression comprising administering the pharmaceutical composition of claim 18.

27. The method of claim 26, wherein the psychiatric disorder is one or more of schizophrenia and negative symptoms of schizophrenia.

28. The method of claim 26, wherein the psychiatric disorder is one or more of major depressive disorder (MDD) and major depressive disorder with mixed features (MDD-MF).

29. The method of claim 26, comprising administering the solid oral dosage form once per day in a total daily amount of between about 100 mg to about 1200 mg of said racemic amisulpride by weight of free base

30. A method of treating a psychiatric disorder comprising:
administering racemic amisulpride as a solid oral dosage form to a subject,
the solid oral dosage form comprising
racemic amisulpride, or pharmaceutically acceptable salts thereof, and
an extended release agent in an amount between about 10% to about 50% by total solid oral dosage form weight;
wherein said administration provides a subject population average maximum QT interval prolongation relative to baseline that is less than about 0.4 milliseconds (ms) per 10 mg of amisulpride.

31. The method of claim 30, wherein the population average maximum QT interval prolongation relative to baseline is the population average maximum QTcF interval prolongation relative to baseline over the time period of 12 hours after said administration.

32. The method of claim 30, wherein the population average maximum QT interval prolongation relative to baseline is less than about 0.35 milliseconds (ms) per 10 mg of amisulpride.

33. The method of claim 30, wherein the population average maximum QT interval prolongation relative to baseline is less than about 0.3 milliseconds (ms) per 10 mg of amisulpride.

34. The method of claim 30, wherein the population average maximum QT interval prolongation relative to baseline is less than about 0.25 milliseconds (ms) per 10 mg of amisulpride.

35. The method of claim 30, wherein said administration is once per day.

36. The method of claim 30, wherein said solid oral dosage form is a tablet.

37. The method of claim 30, wherein said administration is between about 100 mg per day of amisulpride by weight of free base to about 1200 mg per day of amisulpride by weight of free base.

38. The method of claim 37, wherein said psychiatric disorder is selected from schizophrenia, negative symptoms of schizophrenia, persistent depressive disorder (PDD), treatment resistant depression (TRD), major depressive disorder (MDD) and major depressive disorder with mixed features (MDD-MF), and depression.

39. The method of claim 38, wherein the psychiatric disorder is one or more of schizophrenia and negative symptoms of schizophrenia.

40. The method of claim 38, wherein the psychiatric disorder is one or more of major depressive disorder (MDD) and major depressive disorder with mixed features (MDD-MF).

41. The method of claim 30, wherein the extended release agent comprises a matrix forming agent.

42. The method of claim 41, wherein the matrix forming agent comprises one or more cellulosic ethers.

43. The method of claim 30, wherein the extended release agent is in an amount: (a) between about 30% and about 50% by total solid oral dosage form weight: or (b) between about 20% and about 40% by total solid oral dosage form weight: or (c) between about 20% and about 35% by total solid oral dosage form weight.

44. The method of claim 30, wherein the extended release agent comprises hydroxypropyl methylcellulose in an amount between about 20% to about 35% by total solid oral dosage form weight.

45. The method of claim 30, wherein the solid oral dosage form comprises by total solid oral dosage form weight:
between about 35% to about 55% of said racemic amisulpride,
between about 10% to about 50% of a pharmaceutically acceptable filler, and
between about 20% to about 35% of the extended release agent.

46. A pharmaceutical composition in a solid oral dosage form, the solid oral dosage form comprising,
racemic amisulpride, or pharmaceutically acceptable salts thereof; and
an extended release agent in an amount between about 10% to about 50% by total solid
oral dosage form weight,
wherein the solid oral dosage form when dissolution tested using a two-stage in vitro gastrointestinal simulation dissolution test releases less than about 30% of the amisulpride after 1 hour, releases more than about 30% and less than about 55% of the amisulpride after 3 hours, and releases more than about 50% and less than about 90% of the amisulpride after 6 hours.

47. The pharmaceutical composition of claim 46, wherein the solid oral dosage form when dissolution tested using a two-stage in vitro gastrointestinal simulation dissolution test releases less than about 20% of the amisulpride after 1 hour.

48. The pharmaceutical composition of claim 46, wherein the solid oral dosage form when dissolution tested using a two-stage in vitro gastrointestinal simulation dissolution test releases more than about 30% and less than about 50% of the amisulpride after 3 hours.

49. The pharmaceutical composition of claim 46, wherein the solid oral dosage form when dissolution tested using a two-stage in vitro gastrointestinal simulation dissolution test releases more than about 30% and less than about 40% of the amisulpride after 3 hours.

50. The pharmaceutical composition of claim 46, wherein the solid oral dosage form when dissolution tested using a two-stage in vitro gastrointestinal simulation dissolution test releases more than about 50% and less than about 80% of the amisulpride after 6 hours.

51. The pharmaceutical composition of claim 46, wherein the solid oral dosage form when dissolution tested using a two-stage in vitro gastrointestinal simulation dissolution test releases more than about 60% and less than about 90% of the amisulpride after 6 hours.

52. The pharmaceutical composition of claim 46, wherein the two-stage gastrointestinal simulation dissolution test is conducted in a paddle apparatus substantially in accord with that described in Table 4 in the paddle apparatus described in United States Pharmacopeia Convention (USP) Apparatus 2 of Chapter 711 Dissolution; USP41-NF36 General Chapter <711>.

53. The pharmaceutical composition of claim 46, wherein said solid oral dosage form is a tablet.

54. The pharmaceutical composition of claim 53, wherein the amount of racemic amisulpride, or pharmaceutically acceptable salts thereof, is: (a) about 100 mg; or (b) about 150 mg; or (c) about 200 mg; or (d) about 250 mg; or (e) about 300 mg by weight of free base.

55. The pharmaceutical composition of claim 54, wherein the extended release agent comprises a matrix forming agent.

56. The pharmaceutical composition of claim 55, wherein the matrix forming agent comprises one or more cellulosic ethers.

57. The pharmaceutical composition of claim 46, wherein the extended release agent is in an amount: (a) between about 30% and about 50% by total solid oral dosage form weight; or (b) between about 20% and about 40% by total solid oral dosage form weight; or (c) between about 20% and about 35% by total solid oral dosage form weight.

58. The pharmaceutical composition of claim 46, wherein the extended release agent comprises hydroxypropyl methylcellulose in an amount between about 20% to about 35% by total solid oral dosage form weight.

59. A method of treating a psychiatric disorder selected from schizophrenia, negative symptoms of schizophrenia, persistent depressive disorder (PDD), treatment resistant depression (TRD), major depressive disorder (MDD) and major depressive disorder with mixed features (MDD-MF), and depression comprising administering the pharmaceutical composition of claim 46.

60. The method of claim 59, wherein the psychiatric disorder is one or more of schizophrenia and negative symptoms of schizophrenia.

61. The method of claim 59, wherein the psychiatric disorder is one or more of major depressive disorder (MDD) and major depressive disorder with mixed features (MDD-MF).

62. The method of claim 59, comprising administering the solid oral dosage form once per day in a total daily amount of between about 100 mg to about 1200 mg of said racemic amisulpride by weight of free base.

63. A pharmaceutical composition in a solid oral dosage form, the solid oral dosage form comprising,
racemic amisulpride, or pharmaceutically acceptable salts thereof; and
an extended release agent in an amount between about 10% to about 50% by total solid
oral dosage form weight,
wherein the solid oral dosage form when dissolution tested using the two-stage in vitro dissolution test described in Table 4 in the paddle apparatus described in United States Pharmacopeia Convention (USP) Apparatus 2 of Chapter 711 Dissolution; USP41-NF36 General Chapter <711>Dissolution has a dissolution profile substantially the same as: (a) the profile of Tab 1D in FIG. 2; or (b) the profile of Tab 2D in FIG. 2.

64. The pharmaceutical composition of claim 63, wherein said solid oral dosage form is a tablet.

65. The pharmaceutical composition of claim 63, wherein the amount of racemic amisulpride, or pharmaceutically acceptable salts thereof, is: (a) about 100 mg; or (b) about 150 mg; or (c) about 200 mg; or (d) about 250 mg; or (e) about 300 mg by weight of free base.

66. The pharmaceutical composition of claim 63, wherein the extended release agent comprises a matrix forming agent.

67. The pharmaceutical composition of claim 66, wherein the matrix forming agent comprises one or more cellulosic ethers.

68. The pharmaceutical composition of claim 63, wherein the extended release agent is in an amount: (a) between about 30% and about 50% by total solid oral dosage form weight; or (b) between about 20% and about 40% by total solid oral dosage form weight; or (c) between about 20% and about 35% by total solid oral dosage form weight.

69. The pharmaceutical composition of claim 63, wherein the extended release agent comprises hydroxypropyl methylcellulose in an amount between about 20% to about 35% by total solid oral dosage form weight.

70. A method of treating a psychiatric disorder selected from schizophrenia, negative symptoms of schizophrenia, persistent depressive disorder (PDD), treatment resistant depression (TRD), major depressive disorder (MDD) and major depressive disorder with mixed features (MDD-MF), and depression comprising administering the pharmaceutical composition of claim 63.

71. The method of claim 70, wherein the psychiatric disorder is one or more of schizophrenia and negative symptoms of schizophrenia.

72. The method of claim 70, wherein the psychiatric disorder is one or more of major depressive disorder (MDD) and major depressive disorder with mixed features (MDD-MF).

73. The method of claim 70, comprising administering the solid oral dosage form once per day in a total daily amount of between about 100 mg to about 1200 mg of said racemic amisulpride by weight of free base.

74. The solid oral dosage form of any one of claims 1, 18, 30, 46, and 63, wherein the solid oral dosage form comprises one or more of (a) a filler; (b) a binder; and (c) a lubricant.

75. The solid oral dosage form of claim 74, wherein the lubricant comprises magnesium stearate.

76. The solid oral dosage form of claim 74, wherein the filler comprises D-mannitol and wherein the solid oral dosage form comprises between about 0.5% to about 2% by total tablet weight of a binder comprising polyvinyl alcohol.

77. The solid oral dosage form of any one of claims 7, 20, 36, 53, and 64, wherein the tablet comprises:
a granular component admixed with an extra-granular component,
the granular component comprising
the racemic amisulpride and a binder; and
the extra-granular component comprising,
an extended release agent.

78. The pharmaceutical composition of claim 77, wherein the granular component comprises one or more of (a) a filler; and (b) a binder.

79. The pharmaceutical composition of claim 78, wherein the granules comprise: (a) between about 60% to about 80% by weight of amisulpride, between about 10% to about 30% by weight of filler, and between about 1% to about 5% by weight of binder; or (b) between about 70% to about 80% by weight of amisulpride, between about 20% to about 25% by weight of filler, and between about 1% to about 5% by weight of binder.

80. The pharmaceutical composition of claim 78, wherein the granular component comprises:
between about 73% to about 78% by weight of amisulpride,
between about 10% to about 12% by weight of a D-mannitol,
between about 10% to about 12% by weight of a pregelatinized starch, and
between about 1% to about 3% by weight of polyvinyl alcohol;
based on the weight of the granular component.

81. The pharmaceutical composition of claim 77, wherein the extragranular component comprises one or more of (a) a filler; (b) a binder; and (c) a lubricant.

82. The pharmaceutical composition of claim 77, wherein the tablet (granules plus extragranular component) comprises: (a) between about 20% to about 70% by total tablet weight of granules of extended release agent: or (b) between about 10% to about 50% by total tablet weight of extended release agent.

83. The pharmaceutical composition of claim 77, wherein the tablet (granules plus extragranular component) comprises: (a) a combined amount of filler in both granular and extragranular between about 6% to about 60% by total tablet weight; or (b) a combined amount of filler in both granular and extragranular between about 10% to about 50% by total tablet weight.

84. The pharmaceutical composition of claim 77, wherein the tablet (granules plus extragranular component) comprises between about 1% to about 2% by total tablet weight of a lubricant.

85. The pharmaceutical composition of claim 84, wherein the lubricant is magnesium stearate.

86. The pharmaceutical composition of claim 77, wherein the tablet (granules plus extragranular component) comprises:
(a) between about 30% to about 50% by total tablet weight of a D-mannitol, and about 20% to about 35% by total tablet weight of hydroxypropyl methylcellulose; or (b) between about 20% to about 30% by total tablet weight of a D-mannitol, and about 20% to about 35% by total tablet weight of hydroxypropyl methylcellulose; or (c) between about 10% to about 20% by total tablet weight of a D-mannitol, and about 20% to about 35% by total tablet weight of hydroxypropyl methylcellulose.
